(19) United States
(12) Patent Application Publication
(10) Pub. No.: US 2022/0228174 A1
(43) Pub. Date: Jul. 21, 2022

(54) AUTOMATIC START-UP OF ANAEROBIC DIGESTION REACTORS USING MODEL PREDICTIVE CONTROL AND PRACTICALLY FEASIBLE SETS OF MEASUREMENTS

(71) Applicant: Khalifa University of Science and Technology, Abu Dhabi (AE)

(72) Inventors: Jorge RODRIGUEZ, Abu Dhabi (AE); Wasim AHMED, Dubai (AE)

(21) Appl. No.: 17/613,915

(22) PCT Filed: Jun. 24, 2020
(86) PCT No.: PCT/IB2020/055985

§ 371 Date: Nov. 23, 2021

Related U.S. Application Data
(60) Provisional application No. 62/865,875, filed on Jun. 24, 2019.

(51) Int. Cl. C12P 5/02; C12M 1/107; C12M 1/36; C12M 1/34; G16C 20/70

(52) U.S. Cl. CPC C12P 5/023; C12M 21/04; G16C 20/70; C12M 41/46; C12M 41/48

(57) ABSTRACT

Provided is a non-linear model predictive control (NMPC) system for automatic and optimum start-up of an anaerobic digestion (AD) system. The NMPC provides an optimum set of values of manipulated variables for controlling some of the key AD process variables during start-up. The NMPC based automatic start-up system was evaluated against a virtual AD process plant scenario involving a high rate AD reactor treating a readily biodegradable carbohydrate based substrate.

### BACKGROUND

Anaerobic digestion (AD) is a mature, efficient, and renewable biotechnology for organic waste removal/stabilization and/or energy recovery that has been successfully and widely implemented for treatment of a variety of substrates. AD is a biological process in which organic matter is degraded in absence of oxygen to generate methane (CH₄) and CO₂ (biogas) as the major end-products. However, AD involves a complex network of interactions between different groups of micro-organisms that need specific conditions to survive and remain active since they are sensitive to changes in process conditions. For any given AD system, start-up can be a crucial phase as it can determine the entire progression of the system and an ineffective start-up can lead to inefficient process onsets (e.g. sub-optimal or unstable performance in terms of organic matter removal and biogas production). AD start-up can be one of the major operational obstacles owing to the slow growth rate of the key AD microorganisms (particularly methanogens) and the adaptation requirements of the micro-organisms towards the new conditions. Owing to these limitations and in absence of a well-adapted inoculum, AD systems could require long times to start-up (e.g. 2-8 months) before reaching full performance capacity (de Lemos Chernicharo, 2007; Lier et al., 2008; Puñal et al., 2000). For AD systems, reduction of start-up times can be an important factor to increase efficiency and technical competitiveness (de Lemos Chernicharo, 2007) and to minimize costs (Holubar et al., 2003). In this regard, efficient operational control and optimization during AD process start-up can be beneficial and economical in safely driving the system towards an optimal operation (Sbarciog and Vande Wouwer, 2014).

On the practical control side, effective and sufficient monitoring procedures are important for successful operation of an AD process. However, the few number of variables monitored on-line in commercial scale AD systems presents a limitation on the development of effective control strategies. Equipment availability, skilled personnel availability, monitoring costs, and difference in opinions of technical consultants are the major factors affecting the extent of monitoring (e.g. number of variables measured and frequency) implemented in real life AD plants (Drosg, 2013). However, when looking at monitoring costs, one should also evaluate the economic losses resulting from insufficient monitoring (Drosg, 2013). In case of achieving an effective AD start-up, monitoring efforts should be particularly highest since this is a very sensitive phase (Drosg, 2013).

Considering the start-up challenges and limited progress in designing controllers for effective AD start-up management, described herein are improved and practically feasible control processes for automatic start-up of an AD system.

### BRIEF SUMMARY

Provided herein are systems, computer-readable media, and methods for controlling the start-up phase of anaerobic digestion reactors.

In one aspect, the system comprises:

one or more input devices configured at least to receive one or more input signals corresponding to one or more sensors connected with an anaerobic digestion reactor;
one or more output devices configured at least to transmit one or more output signals corresponding to one or more actuators connected with the anaerobic digestion reactor; and
a computing system communicatively connected with the one or more input devices and the one or more output devices, the computing system configured to, at least:
determine, based at least in part on the one or more input signals, one or more values of one or more input variables of a nonlinear model predictive controller, the nonlinear model predictive controller being configured with a nonlinear model of anaerobic digestion having a reduced number of model state variables based at least in part on the one or more input variables that are available due to the one or more input signals;
update the nonlinear model predictive controller based at least in part on the one or more values of the one or more input variables; and
cause the one or more output signals to be generated based at least in part on one or more values of one or more output variables of the nonlinear model predictive controller.

In some embodiments, the nonlinear model of anaerobic digestion comprises a system of ordinary differential equations. In some embodiments, the system of ordinary differential equations is based at least in part on variables corresponding to: an effluent concentration of total acetate from the anaerobic digestion reactor, a concentration of aceticlastic methanogens in the anaerobic digestion reactor, a total alkalinity in the anaerobic digestion reactor, a methane production rate of the anaerobic digestion reactor, an effluent concentration of total inorganic carbon from the anaerobic digestion reactor, an effluent concentration of organic substrate from the anaerobic digestion reactor, and a partial pressure of carbon dioxide in an output of the anaerobic digestion reactor.

In some embodiments, the nonlinear model of anaerobic digestion includes methane production occurring through (i) an aceticlastic methanogenesis pathway and (ii) a hydrogenotrophic methanogenesis pathway.

In some embodiments, the nonlinear model of anaerobic digestion determines total alkalinity from acetate (dissociated), bicarbonate, and hydroxide ions alone.

In some embodiments, the one or more output variables of the nonlinear model predictive controller comprise: a volumetric inflow rate of organic substrate to the anaerobic digestion reactor, a volumetric inflow rate of dilution water to the anaerobic digestion reactor, and a volumetric inflow rate of alkali addition to the anaerobic digestion reactor.

In some embodiments, an objective function of the nonlinear model predictive controller is based at least in part on: an effluent concentration of volatile fatty acids as acetate from the anaerobic digestion reactor, a concentration of aceticlastic methanogens in the anaerobic digestion reactor, a total alkalinity in the anaerobic digestion reactor, a methane production rate of the anaerobic digestion reactor, and a cost term penalizing an amount of alkali added proportional to a volumetric inflow rate of alkali addition to the anaerobic digestion reactor.

In some embodiments, the anaerobic digestion reactor comprises a continuous anaerobic digestion reactor with solids retention.

In some embodiments, the reduced number of model state variables comprises an effluent concentration of total acetate from the anaerobic digestion reactor, a concentration of aceticlastic methanogens in the anaerobic digestion reactor, an effluent concentration of total inorganic carbon from the anaerobic digestion reactor, and a total alkalinity in the anaerobic digestion reactor effluent.

In another aspect, provided are one or more computer-readable media collectively having stored thereon computer-executable instructions.

In some embodiments, the computer-executable instructions, when executed with one or more computing systems, collectively at least:

receive one or more input signals corresponding to one or more sensors connected with an anaerobic digestion reactor;
determine, based at least in part on the one or more input signals, one or more values of one or more input variables of a nonlinear model predictive controller, the nonlinear model predictive controller being configured with a nonlinear model of anaerobic digestion having a reduced number of model state variables based at least in part on the one or more input variables that are available due to the one or more input signals;
update the nonlinear model predictive controller based at least in part on the one or more values of the one or more input variables; and
cause one or more output signals to be generated based at least in part on one or more values of one or more output variables of the nonlinear model predictive controller, the one or more output signals corresponding to one or more actuators connected with the anaerobic digestion reactor

In some embodiments, the nonlinear model of anaerobic digestion comprises a system of ordinary differential equations. In some embodiments, the system of ordinary differential equations is based at least in part on variables corresponding to: an effluent concentration of total acetate from the anaerobic digestion reactor, a concentration of aceticlastic methanogens in the anaerobic digestion reactor, a total alkalinity in the anaerobic digestion reactor, a methane production rate of the anaerobic digestion reactor, an effluent concentration of total inorganic carbon from the anaerobic digestion reactor, an effluent concentration of organic substrate from the anaerobic digestion reactor, and a partial pressure of carbon dioxide in an output of the anaerobic digestion reactor.

In some embodiments, the nonlinear model of anaerobic digestion includes methane production occurring through (i) an aceticlastic methanogenesis pathway and (ii) a hydrogenotrophic methanogenesis pathway.

In some embodiments, the nonlinear model of anaerobic digestion determines total alkalinity from acetate (dissociated), bicarbonate, and hydroxide ions alone.

In some embodiments, the one or more output variables of the nonlinear model predictive controller comprise: a volumetric inflow rate of organic substrate to the anaerobic digestion reactor, a volumetric inflow rate of dilution water to the anaerobic digestion reactor, and a volumetric inflow rate of alkali addition to the anaerobic digestion reactor.

In some embodiments, an objective function of the nonlinear model predictive controller is based at least in part on: an effluent concentration of volatile fatty acids as acetate from the anaerobic digestion reactor, a concentration of aceticlastic methanogens in the anaerobic digestion reactor, a methane production rate of the anaerobic digestion reactor, and a cost term penalizing an amount of alkali added proportional to a volumetric inflow rate of alkali addition to the anaerobic digestion reactor.

In some embodiments, the anaerobic digestion reactor comprises a continuous anaerobic digestion reactor with solids retention.

In some embodiments, the reduced number of model state variables comprises an effluent concentration of total acetate from the anaerobic digestion reactor, a concentration of aceticlastic methanogens in the anaerobic digestion reactor, an effluent concentration of total inorganic carbon from the anaerobic digestion reactor, and a total alkalinity in the anaerobic digestion reactor effluent.

In another aspect, a method for controlling a start-up phase of anaerobic digestion reactor operation is provided, the method comprising:

receiving, with one or more input devices, one or more input signals corresponding to one or more sensors connected with an anaerobic digestion reactor;
determining, with a computing system, based at least in part on the one or more input signals, one or more values of one or more input variables of a nonlinear model predictive controller, the nonlinear model predictive controller being configured with a nonlinear model of anaerobic digestion having a reduced number of model state variables based at least in part on the one or more input variables that are available due to the one or more input signals;
updating, with the computing system, the nonlinear model predictive controller based at least in part on the one or more values of the one or more input variables; and
causing, with the computing system, one or more output signals to be generated based at least in part on one or more values of one or more output variables of the nonlinear model predictive controller, the one or more output signals corresponding to one or more actuators connected with the anaerobic digestion reactor.

In some embodiments of the method, the nonlinear model of anaerobic digestion comprises a system of ordinary differential equations. In some embodiments, the system of ordinary differential equations is based at least in part on variables corresponding to: an effluent concentration of total acetate from the anaerobic digestion reactor, a concentration of aceticlastic methanogens in the anaerobic digestion reactor, a total alkalinity in the anaerobic digestion reactor, a methane production rate of the anaerobic digestion reactor, an effluent concentration of total inorganic carbon from the anaerobic digestion reactor, an effluent concentration of organic substrate from the anaerobic digestion reactor, and a partial pressure of carbon dioxide in an output of the anaerobic digestion reactor.

In some embodiments, the nonlinear model of anaerobic digestion includes methane production occurring through (i) an aceticlastic methanogenesis pathway and (ii) a hydrogenotrophic methanogenesis pathway.

In some embodiments, wherein the nonlinear model of anaerobic digestion determines total alkalinity from acetate (dissociated), bicarbonate, and hydroxide ions alone.

In some embodiments, the one or more output variables of the nonlinear model predictive controller comprise: a volumetric inflow rate of organic substrate to the anaerobic digestion reactor, a volumetric inflow rate of dilution water to the anaerobic digestion reactor, and a volumetric inflow rate of alkali addition to the anaerobic digestion reactor.

In some embodiments, an objective function of the nonlinear model predictive controller is based at least in part on: an effluent concentration of volatile fatty acids as acetate from the anaerobic digestion reactor, a concentration of aceticlastic methanogens in the anaerobic digestion reactor, a methane production rate of the anaerobic digestion reactor, and a cost term penalizing an amount of alkali added proportional to a volumetric inflow rate of alkali addition to the anaerobic digestion reactor.

In some embodiments, the anaerobic digestion reactor comprises a continuous anaerobic digestion reactor with solids retention.

In some embodiments, the reduced number of model state variables comprises an effluent concentration of total acetate in the anaerobic digestion reactor, a concentration of aceticlastic methanogens from the anaerobic digestion reactor, an effluent concentration of total inorganic carbon from the anaerobic digestion reactor, and a total alkalinity in the anaerobic digestion reactor effluent.

### BRIEF DESCRIPTION OF THE DRAWINGS

FIG. 1 shows a schematic of an exemplary model predictive control (MPC) concept.

FIG. 2 shows an exemplary nonlinear model predictive control (NMPC) scheme for optimal start-up control of an AD system.

FIG. 3 shows the bioconversion pathways incorporated in the exemplary simple AD model (SADM) described herein.

FIG. 4 shows an exemplary SADM predictions block interface with process variables information from an AD system or plant.

FIG. 5 is a schematic diagram illustrating an example operating environment in accordance with at least one embodiment.

FIG. 6 is a flow diagram illustrating an example procedure in accordance with at least one embodiment.

FIG. 7 is a schematic diagram illustrating an example computing system in accordance with at least one embodiment.

FIG. 8 shows an exemplary aceticlastic methanogenic biomass concentration (control variable) estimation methodology (measurement delay duration arbitrarily set for illustrative purpose only).

FIGS. 9(a)-(i) show exemplary AD start-up performance and operation with NMPC strategies (NMPC Base and NMPC No rCH4) in comparison with manual strategies. FIGS. 9 (a)-(c) show dilution rates of organic substrate, dilution water and alkali input respectively (MVs). FIGS. 9 (d)-(f) show effluent acetate, reactor concentration of aceticlastic methanogenic biomass, and CH₄ production rate respectively (CVs). FIG. 9(g) shows applied organic loading rate (OLR). FIG. 9(h) shows pH of the AD (virtual) plant. FIG. 9(i) shows the Alkalinity ratio (intermediate to total alkalinity) of the AD (virtual) plant.

FIGS. 10(a) and (b) show the values of the individual cost terms in the overall objective functions of the two proposed NMPC designs (Eq. (11) for NMPC Base and Eq. (19) for NMPC_{No rCH4} in the journal article) after optimisation at each sampling instant for the AD start-up case scenario considered in the journal article. The durations for the control designs in FIG. 10 represent the periods during which the controllers were active. FIG. 10(a) and FIG. 10(b) show the resulting values of the cost terms with NMPC_{Base} and NMPC_{No rCH4} control designs respectively (S_{ac}-effluent concentration of VFAs as acetate; X_{ac}—reactor concentration of aceticlastic methanogenic biomass; _{rCH4}—CH₄ production rate).

FIGS. 11 and 12 compare the sADM (prediction model during NMPC optimisations) predictions of process variables with those of the virtual plant (modelled via ADM1) during the NMPC scheme implementations (NMPC_{Base} and NMPC_{No rCH4} respectively). The durations in FIGS. 11-13 represent the time periods during which the NMPC controllers were active.

FIGS. 11(a)-(d) show a comparison between sADM and virtual plant (ADM1) predictions of AD process variables during implementation of NMPC Base: FIG. 11(a) effluent concentration of VFAs (as acetate), FIG. 11(b) reactor concentration of aceticlastic methanogenic biomass, FIG. 11(c) CH4 production rate, and FIG. 11(d) pH.

FIGS. 12(a)-(c) show a comparison between sADM and virtual plant (ADM1) predictions of AD process variables during implementation of NMPC No rCH4: (a) effluent concentration of VFAs (as acetate), (b) reactor concentration of aceticlastic methanogenic biomass, and (c) pH.

FIGS. 13(a) and 13(b) compare the Estimator/Observer estimation vs. virtual plant prediction (ADM1) of aceticlastic methanogenic biomass in reactor during NMPC simulations: FIG. 13(a) NMPC_{Base}; FIG. 13(b) NMPC_{No rCH4}.

FIGS. 14(a)-(f) show the performance of the proposed NMPC system on the start-up of an AD (ADM1-based virtual) plant under NMPC Base considering model mismatch/bias correction. FIGS. 14(a)-(c) MVs of dilution rates for organic substrate, dilution water and concentrated alkali respectively; subfigure in FIG. 14(a) OLR. FIGS. 14(d)-(f) CVs from the AD (virtual) plant of effluent acetate and reactor aceticlastic methanogenic biomass concentrations, and methane production rate respectively.

FIGS. 15(a)-(f) show the performance of the proposed NMPC system on the start-up of an AD (ADM1-based virtual) plant under NMPC_{No rCH4} considering model mismatch/bias correction. FIGS. 15(a)-(c) MVs of dilution rates for organic substrate, dilution water and concentrated alkali respectively; subfigure in (a) OLR. FIGS. 15(d)-(f) CVs from the AD (virtual) plant of effluent acetate and reactor aceticlastic methanogenic biomass concentrations, and methane production rate respectively.

FIGS. 16(a)-(f) show the performance of the proposed NMPC system on the start-up of an AD (ADM1-based virtual) plant under NMPC_{Base} assuming online measurement of X_{ac}. FIGS. 16(a)-(c) MVs of dilution rates for organic substrate, dilution water and concentrated alkali respectively; subfigure in (a) OLR. FIGS. 16(d)-(f) CVs from the AD (virtual) plant of effluent acetate and reactor aceticlastic methanogenic biomass concentrations, and methane production rate respectively.

FIGS. 17(a)-(f) show the performance of the proposed NMPC system on the start-up of an AD (ADM1-based virtual) plant under NMPC_{No rCH4} assuming online measurement of X_{ac}.

FIGS. 17 (a)-(c) MVs of dilution rates for organic substrate, dilution water and concentrated alkali respectively; subfigure in (a) OLR. FIGS. 17 (d)-(f) CVs from the AD (virtual) plant of effluent acetate and reactor aceticlastic methanogenic biomass concentrations, and methane production rate respectively.

FIGS. 18 (a)-(f) show the performance of the proposed NMPC system on the start-up of an AD (ADM1-based virtual) plant under NMPC_{Base} in presence of measurement errors (random errors ranging between −10% and 10% relative to actual measurements). FIGS. 18(a)-(c) MVs of dilution rates for organic substrate, dilution water and concentrated alkali respectively; subfigure in (a) OLR. FIGS. 18(d)-(f) CVs from the AD (virtual) plant of effluent acetate and reactor aceticlastic methanogenic biomass concentrations, and methane production rate respectively.

FIGS. 19 (a)-(f) show the performance of the proposed NMPC system on the start-up of an AD (ADM1-based virtual) plant under NMPC_{No rCH4} in presence of measurement errors (random errors ranging between −10% and 10% relative to actual measurements). FIGS. 19(a)-(c) MVs of dilution rates for organic substrate, dilution water and concentrated alkali respectively; subfigure in (a) OLR. FIGS. 19(d)-(f) CVs from the AD (virtual) plant of effluent acetate and reactor aceticlastic methanogenic biomass concentrations, and methane production rate respectively.

FIGS. 20(a)-(f) show the performance of the proposed NMPC system on the start-up of an AD (ADM1-based virtual) plant under NMPC_{Base} in presence of measured disturbance of substrate influent COD (0.69 gCOD/L at 10^{th} day for 15 days). FIGS. 20(a)-(c) MVs of dilution rates for organic substrate, dilution water and concentrated alkali respectively; subfigure in (a) OLR. FIGS. 20(d)-(f) CVs from the AD (virtual) plant of effluent acetate and reactor aceticlastic methanogenic biomass concentrations, and methane production rate respectively.

FIGS. 21(a)-(f) show the performance of the proposed NMPC system on the start-up of an AD (ADM1-based virtual) plant under NMPC_{No rCH4} in presence of measured disturbance of substrate influent COD (0.69 gCOD/L at 10^{th} day for 15 days). FIGS. 21(a)-(c) MVs of dilution rates for organic substrate, dilution water and concentrated alkali respectively; subfigure in (a) OLR. FIGS. 21(d)-(f) CVs from the AD (virtual) plant of effluent acetate and reactor aceticlastic methanogenic biomass concentrations, and methane production rate respectively.

FIGS. 22(a)-(f) show the performance of the proposed NMPC system on the start-up of an AD (ADM1-based virtual) plant under NMPC_{Base} in presence of measured disturbance of substrate influent COD (4.8 gCOD/L at 10^{th} day for 15 days). FIGS. 22(a)-(c) MVs of dilution rates for organic substrate, dilution water and concentrated alkali respectively; subfigure in (a) OLR. FIGS. 22(d)-(f) CVs from the AD (virtual) plant of effluent acetate and reactor aceticlastic methanogenic biomass concentrations, and methane production rate respectively.

FIGS. 23(a)-(f) show the performance of the proposed NMPC system on the start-up of an AD (ADM1-based virtual) plant under NMPC_{No rCH4} in presence of measured disturbance of substrate influent COD (4.8 gCOD/L at 10^{th} day for 15 days). FIGS. 23(a)-(c) MVs of dilution rates for organic substrate, dilution water and concentrated alkali respectively; subfigure in (a) OLR. FIGS. 23(d)-(f) CVs from the AD (virtual) plant of effluent acetate and reactor aceticlastic methanogenic biomass concentrations, and methane production rate respectively.

FIGS. 24(a)-(f) show the performance of the proposed NMPC system on the start-up of an AD (ADM1-based virtual) plant under NMPC_{Base} in presence of unmeasured disturbance of substrate influent COD (random changes ranging between −5% and 5% relative to base influent COD of 2.75 gCOD/L). FIGS. 24(a)-(c) MVs of dilution rates for organic substrate, dilution water and concentrated alkali respectively; subfigure in (a) OLR. FIGS. 24(d)-(f) CVs from the AD (virtual) plant of effluent acetate and reactor aceticlastic methanogenic biomass concentrations, and methane production rate respectively.

FIGS. 25(a)-(f) show the performance of the proposed NMPC system on the start-up of an AD (ADM1-based virtual) plant under NMPC_{No rCH4} in presence of unmeasured disturbance of substrate influent COD (random changes ranging between −5% and 5% relative to base influent COD of 2.75 gCOD/L). FIGS. 25(a)-(c) MVs of dilution rates for organic substrate, dilution water and concentrated alkali respectively; subfigure in (a) OLR. FIGS. 25(d)-(f) CVs from the AD (virtual) plant of effluent acetate and reactor aceticlastic methanogenic biomass concentrations, and methane production rate respectively.

FIGS. 26(a)-(f) show the performance of the proposed NMPC system on the start-up of an AD (ADM1-based virtual) plant under NMPC_{Base} in presence of unmeasured disturbance of substrate influent COD (random changes ranging between −75% and 75% relative to base influent COD of 2.75 gCOD/L). FIGS. 26(a)-(c) MVs of dilution rates for organic substrate, dilution water and concentrated alkali respectively; subfigure in (a) OLR. FIGS. 26(d)-(f) CVs from the AD (virtual) plant of effluent acetate and reactor aceticlastic methanogenic biomass concentrations, and methane production rate respectively.

FIGS. 27(a)-(f) show the performance of the proposed NMPC system on the start-up of an AD (ADM1-based virtual) plant under NMPC_{No rCH4} in presence of unmeasured disturbance of substrate influent COD (random changes ranging between −75% and 75% relative to base influent COD of 2.75 gCOD/L). FIGS. 27(a)-(c) MVs of dilution rates for organic substrate, dilution water and concentrated alkali respectively; subfigure in (a) OLR. FIGS. 27(d)-(f) CVs from the AD (virtual) plant of effluent acetate and reactor aceticlastic methanogenic biomass concentrations, and methane production rate respectively.

FIGS. 28(a)-(f) show the performance of the proposed NMPC system on the start-up of an AD (ADM1-based virtual) plant under NMPC_{Base} in presence of unmeasured disturbance of substrate influent COD (0.55 gCOD/L at 10^{th} day for 15 days). FIGS. 28(a)-(c) MVs of dilution rates for organic substrate, dilution water and concentrated alkali respectively; subfigure in (a) OLR. FIGS. 28(d)-(f) CVs from the AD (virtual) plant of effluent acetate and reactor aceticlastic methanogenic biomass concentrations, and methane production rate respectively.

FIGS. 29(a)-(f) show the performance of the proposed NMPC system on the start-up of an AD (ADM1-based virtual) plant under NMPC_{No rCH4} in presence of unmeasured disturbance of substrate influent COD (0.55 gCOD/L at 10^{th} day for 15 days). FIGS. 29(a)-(c) MVs of dilution rates for organic substrate, dilution water and concentrated alkali respectively; subfigure in (a) OLR. FIGS. 29(d)-(f) CVs from the AD (virtual) plant of effluent acetate and reactor aceticlastic methanogenic biomass concentrations, and methane production rate respectively.

FIGS. 30(a)-(f) show the performance of the proposed NMPC system on the start-up of an AD (ADM1-based virtual) plant under NMPC_{Base} in presence of unmeasured disturbance of substrate influent COD (5.0 gCOD/L at 10^{th} day for 15 days). FIGS. 30(a)-(c) MVs of dilution rates for organic substrate, dilution water and concentrated alkali respectively; subfigure in (a) OLR. FIGS. 30(d)-(f) CVs from the AD (virtual) plant of effluent acetate and reactor aceticlastic methanogenic biomass concentrations, and methane production rate respectively.

FIGS. 31(a)-(f) show the performance of the proposed NMPC system on the start-up of an AD (ADM1-based virtual) plant under NMPC_{No rCH4} in presence of unmeasured disturbance of substrate influent COD (5.0 gCOD/L at 10^{th} day for 15 days). FIGS. 31(a)-(c) MVs of dilution rates for organic substrate, dilution water and concentrated alkali respectively; subfigure in (a) OLR. FIGS. 31(d)-(f) CVs from the AD (virtual) plant of effluent acetate and reactor aceticlastic methanogenic biomass concentrations, and methane production rate respectively.

### DEFINITIONS

### Nomenclature

A_{reactor} Cross-sectional area of reactor (m²)
Cₐₗₖ Concentration of alkali input stream (g alkali/L)
D Dilution rate (1/h)
HRT Hydraulic retention time (h)
(IA/TA)max Maximum intermediate alkalinity to total alkalinity ratio
I_{pH} pH inhibition function
J_{P} Objective function over prediction horizon (dimensionless)
k Sampling time instant (where at each sampling instant, t=k*Ts)
k_{d} Biomass decay rate constant (1/h)
k_{L}a Liquid to gas mass transfer coefficient for CO₂ (1/h)
K_{H, CO}_{₂} Henry's constant for CO₂ (M/bar)
K_{S} Half-saturation constant for aceticlastic methanogenesis (M)
M Control horizon (number of sampling time steps)
M_{COD,ac} Molar chemical oxygen demand (COD) of acetate (gCOD/mol acetate)
M_{COD,bu} Molar COD of butyrate (gCOD/mol butyrate)
M_{COD,CH}_{₄} Molar COD of CH₄ (gCOD/mol CH₄)
M_{COD,H}_{₂} Molar COD of H₂ (gCOD/mol H₂)
M_{COD,pro} Molar COD of propionate (gCOD/mol propionate)
M_{COD,S} Molar COD of organic substrate (gCOD/mol substrate)
M_{COD,pro} Molar COD of valerate (gCOD/mol valerate)
OLR Organic loading rate (kgCOD/m³·d or gCOD/L·h)
OLRₘₐₓ Maximum organic loading rate (gCOD/L·h)
P Prediction horizon (number of sampling time steps)
P_{CO}_{₂}(t) CO₂ partial pressure from AD plant output (bar)
qₘ Maximum specific uptake rate for aceticlastic methanogenesis (mol Ac/mol X·h)
Qₐₗₖ Volumetric inflow rate of alkali addition (L/h)
Q_{H}_{₂}_{O} Volumetric inflow rate of dilution water (L/h)
Q_{S} Volumetric inflow rate of organic substrate (L/h)
r_{ac,m} Acetate uptake rate for aceticlastic methanogenesis (mol Ac/L·h)
r_{CH}_{₄} CH₄ production rate (gCOD/L_{reactor}·h)
r_{H}_{₂}_{,m} H₂ uptake rate for hydrogenotrophic methanogenesis (mol H₂/L·h)
r_{S}_{_{S}}(t) Organic substrate degradation/uptake rate (mol S/L·h)
S_{ac} Effluent concentration of total acetate (M)
S_{ac,eq} VFA concentration in acetate equivalents (M)
S_{ac,inf} Influent concentration of total acetate (M)
S_{bu} Effluent/reactor concentration of total butyrate (M)
S_{CH4} Effluent/reactor concentration of methane in liquid phase (M)
S_{CO}_{₂}_{. aq} Effluent/reactor concentration of aqueous CO₂ (M)
S_{IC} Effluent/reactor concentration of total inorganic carbon (M)
S_{IC,inf} Influent concentration of total inorganic carbon (M)
Sₚᵣₒ Effluent/reactor concentration of total propionate (M)
Ssu Effluent/reactor concentration of sugars (M)
Sᵥₐ Effluent/reactor concentration of total valerate (M)
SRT Solids retention time (h)
Sₛ (t) Effluent concentration of organic substrate from AD plant (M)
S_{s,inf} Influent concentration of organic substrate (M)
t Time (h)
Ts Sampling time (h)
V_{reactor} or Vᵣ Liquid volume of reactor (L)
νᵤₚ^{max} Maximum liquid upflow velocity (m/h)
νᵤₚ^{min} Minimum liquid upflow velocity (m/h)
w₁, w₂, w₃, w₄ NMPC objective function weights
X Simple AD model (SADM) states
X_{ac} Concentration of aceticlastic methanogens in reactor (M)
\tilde{Y}ₖ Predicted control variables at sampling time instant k
Yₖ^{sp} Control variable set-point at sampling time instant k
Y_{X, cumulative} Cumulative biomass yield for overall organic substrate-to-methane reaction (mol X/mol Ss)
Yₓ_{_{ac}}_{/ac} Biomass yield of aceticlastic methanogens (mol X/mol Ac)
Z Total alkalinity in reactor/effluent
Z_{inf} Total alkalinity in influent

Greek letters
ν_{ac/S} Stoichiometric yield of acetate from organic substrate degradation (mol Ac/mol substrate)
ν_{CO}_{₂}_{/ac} Stoichiometric yield of CO₂ from aceticlastic methanogenesis (mol CO₂/mol substrate)
ν_{CO}_{₂}_{/s} Stoichiometric yield of CO₂ from organic substrate degradation (mol CO₂/mol substrate)
ν_{CO}_{₂}_{/H}_{₂} Stoichiometric coefficient of CO₂ consumption via hydrogenotrophic methanogenesis (mol CO₂/mol H₂)
ν_{CH}_{₄}_{/ac} Stoichiometric yield of CH₄ from aceticlastic methanogenesis (gCOD-CH₄/gCOD-Ac)
ν_{CH}_{₄}_{/H}_{₂} Stoichiometric yield of CH₄ from hydrogenotrophic methanogenesis (gCOD-CH₄/gCOD-H₂)
ν_{H}_{₂}_{/S} Stoichiometric yield of H₂ from organic substrate degradation (mol H₂/mol substrate)
ρ_{T,ch4} Liquid to gas mass transfer rate of CH₄ (mol/L_{reactor}·h)

Subscript
k Sampling time instant
LB Lower bound

Superscript
sp Set-point

As used herein, “methanogenesis” refers to the formation of methane by microbes known as methanogens.

As used herein, a “methanogen” refers to a microorganisms that produces methane as a metabolic byproduct under anaerobic conditions.

As used herein, “acetoclastic methanogenesis” refers to methanogenesis from acetate.

As used herein, “acetoclastic methanogen” refers to a microorganism that produces methane from acetate, such as methanogenic euryarchaea of the order Methanosarcinales.

### DETAILED DESCRIPTION

Described herein is a nonlinear model predictive control (NMPC) scheme for automatic (and ideally optimal) start-up of an anaerobic digestion (AD) system. The control scheme is designed with its practical implementation in mind, based on the use of feasible-to-measure process variables and a very condensed simple AD model (as a prediction model for NMPC optimizations). An overall comprehensive control framework is provided as a practically feasible tool for operators and interested users to implement. The simple AD model (SADM) implemented in the NMPC scheme provides the advantage of predictions of the complex AD process with practical usability and minimal structural complexities. The SADM described herein provides advantages over existing models. Example advantages include decreasing the number of model states and incorporating both methanogenesis pathways for a justified and more accurate prediction of CH₄ output. As another example, the SADM also provides the advantage of interfacing with an existing AD plant system to use the available measurements and system updates for estimation of the main model variables and rates, such that the number of model states and complexity remain small and simple. The model thus provides a practical feature that is useful for AD plant operators and engineers. This can enable future operations of the AD plant to be modeled and thereby help predict future operational issues and direct early corrective action using variables provided by the existing AD plant.

In one aspect, the model is useful for AD of readily biodegradable substrates in continuous reactor systems with solids retention. The results of NMPC simulations show that the SADM predictions are generally in good agreement with virtual AD plant behavior (e.g., modelled with ADM1 which is one of the most comprehensive AD models and a widely known and accepted model for AD processes).

There are a number of factors affecting the AD start-up phase such as feed characteristics (type, composition, and concentration), seed sludge or inoculum characteristics (quality, microbial community profile, and relative proportion of micro-organisms with respect to each other), feed loading rates, hydraulic retention time (HRT), and solids retention time (SRT) (Goberna et al., 2015; Janke et al., 2016; Pandey et al., 2011; Vadlani and Ramachandran, 2008). Several experimental studies (focusing on operational aspects), ranging from lab scale to full scale systems testing, exist in literature that have focused on one or more of these factors to propose strategies for effective and/or rapid start-up of AD systems (Alvarado-Lassman et al., 2010; Angelidaki et al., 2006; Angenent et al., 2002; Borzacconi et al., 2006; Dong et al., 2010; Fdéz.-Güelfo et al., 2010; Goux et al., 2016; Janke et al., 2016; Lagerkvist et al., 2015; Liang et al., 2017; Lloret et al., 2013; Meng et al., 2014; Pandey et al., 2011; Puñal et al., 2000; Vadlani and Ramachandran, 2008; Williams et al., 2013). Based on these studies, AD start-up phase has been typically considered complete after achievement of one or more of the following: design organic loading rates (OLR); stable and non-inhibitory levels of volatile fatty acids (VFAs) and/or pH; adequate substrate removal efficiencies; stable biogas production (in terms of flow, CH₄ content, and/or composition).

In terms of operational strategy, feeding regime (e.g. OLR and nutrients supply) and inoculation procedures (e.g. inoculum availability, characteristics, and amount) have been major factors affecting the AD performance during start-up according to the existing experimental studies on AD start-up. The feeding regime during AD start-up allows the sensitive micro-organisms to adapt to the different or changing environments and also to avoid any destabilizations in case of feed overloads and/or acidification inside the reactor. As such, it is commonly observed across the existing studies that the AD systems are started up, after carrying out any necessary inoculation procedures, by feeding in a gradual manner i.e. the OLR is low initially and then gradually increased according to the status of the digester (e.g. VFA levels, pH, and biogas production). Although the results have been successful, such manual operational feeding strategies provide no assurance of optimality and are likely suboptimal. Fearing AD process destabilization, some plant operators reject valuable feed (Holubar et al., 2003) during start-up phase. Also, according to some technical reports and guides on biogas plant monitoring and operation, starting up with significantly low organic loads is not advised since it can lower AD plant productivity and negatively impact microbial growth due to lack of adequate food supply (Drosg, 2013; Esteves et al., 2012).

Aiming for optimal operation during AD start-up can help in increasing the practical feasibility of AD technology at large scale. Model predictive control (MPC) provides a strategy for improving AD start-up, where model predictions and latest measurements are used to determine the set of control input or manipulated variable (MV) changes that can optimally drive the control outputs or variables (CVs) to their desired targets (set-points) (Seborg et al., 2011). A characteristic feature of MPC contributing to its success is the consideration of constraints while carrying out the control optimizations (Grune and Pannek, 2011). Very few studies exist in literature on MPC implementation for AD systems (Gaida et al., 2012, 2011; Haugen et al., 2014; Kil et al., 2017; Mauky et al., 2016; Ordace et al., 2012; Xue et al., 2015), and the inventors are unaware of MPC studies particularly focusing on AD start-up. Nevertheless, these studies have shown the effectiveness of MPC strategy for controlling AD systems and its flexibility in implementing different control designs and scenarios.

A factor in the success of model based controllers including MPC is the availability of an accurate and reliable model. For virtual testing scenarios prior to experimental testing, using reliably structured and complex AD models as virtual plants is important for accurate representation of the process plant behavior. With accurate virtual plant models, the control schemes can be more accurately evaluated and assessed. In some embodiments, the comprehensive and widely accepted Anaerobic Model No. 1 (ADM1) by Batstone et al. (2002) is used as the virtual plant.

### Model Predictive Control (MPC) Basic Principles

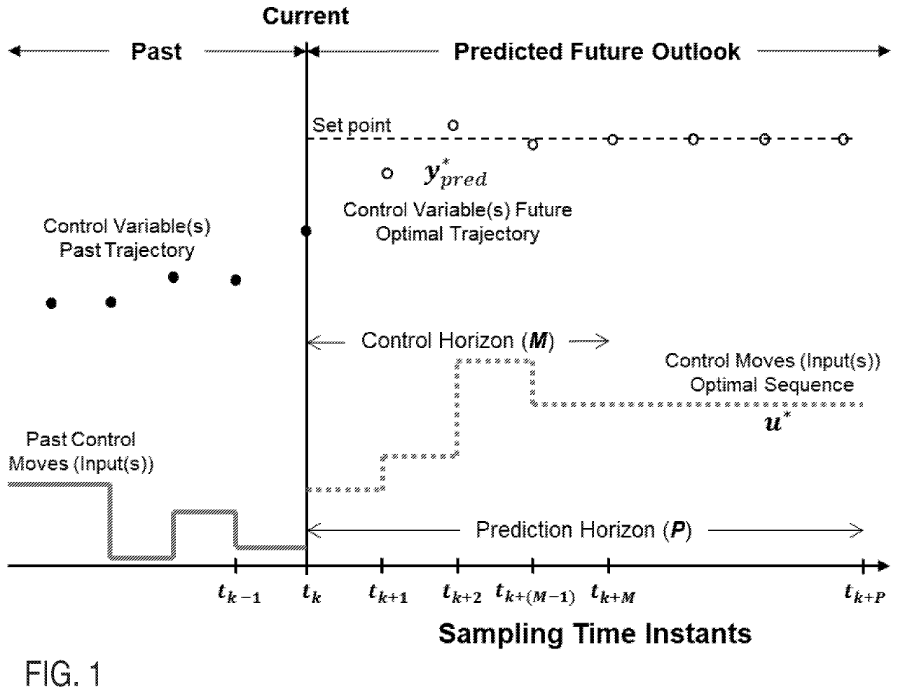

The main objective of conventional MPC is to determine a set of manipulated input changes (i.e. MVs) to optimally drive the selected process outputs (i.e. CVs) to their desired set points (Seborg et al., 2011) while considering constraints (if any). These optimization calculations incorporate process plant measurements and model predicted future outlook of the outputs. FIG. 1 shows one example of a MPC. At the current sampling time instant (tₖ), using latest available measurements and model for predictions of future outputs, MPC calculates an optimal set of control moves or input values (u*) that can be manipulated (i.e. MVs) within an allowable number of time steps for control, specifically called the control horizon (M). After the reaching the control horizon, the input values are fixed at a constant value (equal to the input values determined for the last time step within the control horizon i.e. at t_{k+(M-1)}) for the remaining time steps till the prediction horizon (P). Although a sequence of control moves over the entire prediction horizon (P) is determined, MPC implements only the first time step control moves set. These control calculations are repeated at each sampling time instant. In some embodiments, the basic MPC algorithm can be summarized as follows (Grune and Pannek, 2011):

At each sampling time instant,
1. Measure outputs and states of the system
2. Using the latest measurements as the initial states, solve the optimal control problem to optimize the desired control objective function over the prediction horizon (P) and consequently, determine the optimal sequence of control inputs (size=number of MVs x control horizon)
3. Select the first time step input set from the sequence and implement it on the process plant being controlled.

The adopted process model can be utilized in parallel with MPC calculations. While a linear or linearized model based MPC (linear MPC) is common, using a nonlinear model can be advantageous in case of highly nonlinear systems (Seborg et al., 2011). Thus, in some embodiments, the control scheme is a nonlinear MPC (NMPC) scheme.

### NMPC Scheme Architecture for Optimal and Automatic Ad Start-Up

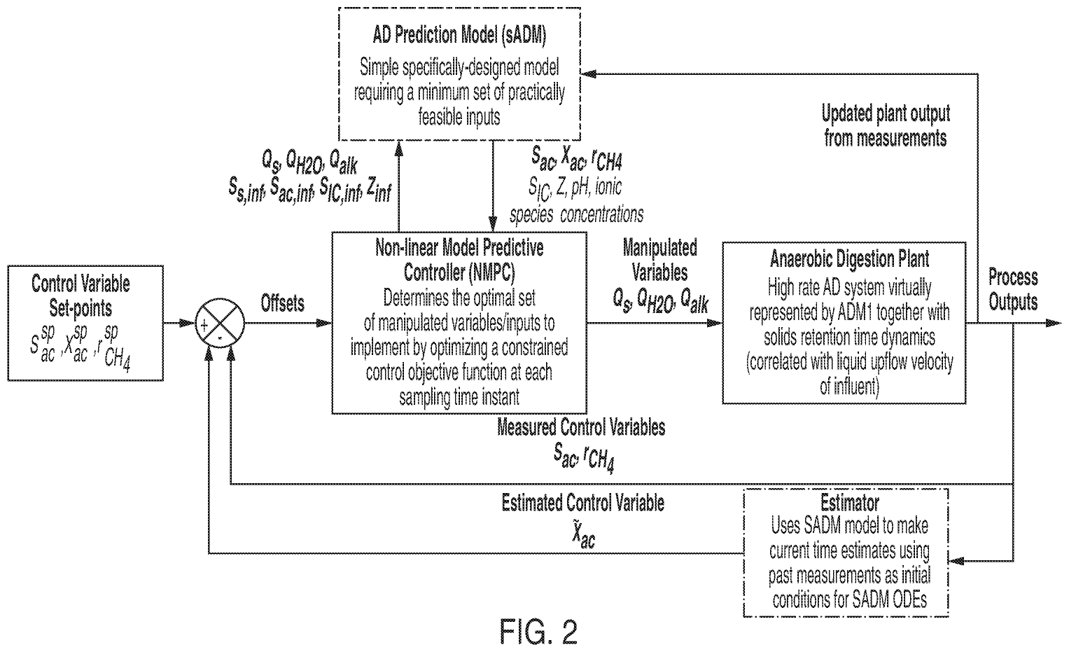

An exemplary NMPC scheme for optimal start-up control is depicted in FIG. 2. The general MPC concept is described in Appendix A. In some embodiments, the control scheme involves regulating two-three control variables (CVs) around set-points: 1) effluent concentration of volatile fatty acids (VFAs) as acetate (S_{ac}), 2) concentration of aceticlastic methanogens in reactor (X_{ac}), and 3) CH₄ production rate (r_{CH4}) as an additional CV for one of the test scenarios implemented in this work. For regulating the CVs, three manipulated variables (MVs) or control inputs are considered: volumetric inflow rates of 1) organic substrate (Q_{S}), 2) dilution water (Q_{H}_{₂}_{O}), and 3) concentrated alkali input (Qₐₗₖ). In some embodiments of the model based control scheme describe herein, a simple AD model (SADM) is used as the prediction model and it is developed in a manner where information from available measurements in AD systems is used. With the measured and estimated process outputs (CVs and other outputs) providing feedback on the latest state of the AD process after every sampling time, the NMPC uses model predictions from SADM to optimize a desired control objective function (within constraints) and consequently, finds the optimum set of MVs to implement on the AD system/plant (virtually represented by ADM1 in current work). In this way, the NMPC is aimed at regulating the CVs around the set-points (X_{ac}^{sp}, S_{ac}^{sp}, r_{CH}_{₄}^{sp}) while considering constraints.

By incorporating the CVs in the NMPC scheme (FIG. 2), the following factors are addressed during start-up: 1) effluent quality, 2) growth of methanogens (particularly aceticlastic group), and 3) actual chemical oxygen demand (COD) removal via methanation. Chemical oxygen demand (COD) removal efficiency is an indicator of effluent quality in terms of organic matter degradation and achievement of a certain removal efficiency is one of the indicators of start-up completion. With this regard, the levels of volatile fatty acid (VFA) product intermediates in reactor effluent, particularly acetate (one of the key precursors for methanogenesis), can be helpful. Also, VFA levels can give an indication of process stability in terms of the level of acidification inside the reactor. In some embodiments, the AD start-up comprises concentrating microbial biomass or favoring their growth (see Lier et al. (2008). These micro-organisms are responsible for driving the AD process and information on their abundance and/or diversity gives a direct indication on the status of the digester performance. In some embodiments, the microbial population profiles are monitored to provide an assessment of the digester status, which allows a plant operator to better predict process dynamics and take decisive operational management activities.

In some embodiments, aceticlastic methanogenic biomass is an effective target group for favoring methanogenic biomass capacity build-up in the reactor and provides a novel CV in the NMPC control scheme for AD start-up described herein. Aceticlastic methanogenic micro-organisms have slow growth rates (maximum specific growth rates of 0.12-2.85 1/d (Lier et al., 2008)), are highly sensitive to a number of conditions (e.g. pH, temperature, ammonia levels), and are major CH₄ producers amongst the methanogenic groups.

Production of CH₄, together with the levels of VFA intermediates, confirms the conversion and removal of COD from the system. In some embodiments, methane production is one of the common variables measured and monitored for assessing AD output performance. In some embodiments, methane production can be used to indirectly assess stability in AD systems.

In some embodiments, adjusting the organic substrate flow rate during start-up is a manipulated variable (MV). Dilution water can be used to reduce process acidification by diluting the accumulating VFAs. Also, such dilution can help to temporarily reduce organic levels in case of unexpected organic feed overloads. In some embodiments, additional alkali is added to increase the buffering capacity of the AD system and avoid acidification. Acid overload can be a common problem during start-up (e.g., see Lagerkvist et al., 2015), typically when treating readily biodegradable substrates.

### Simple AD Model (SADM) Structure for NMPC Scheme

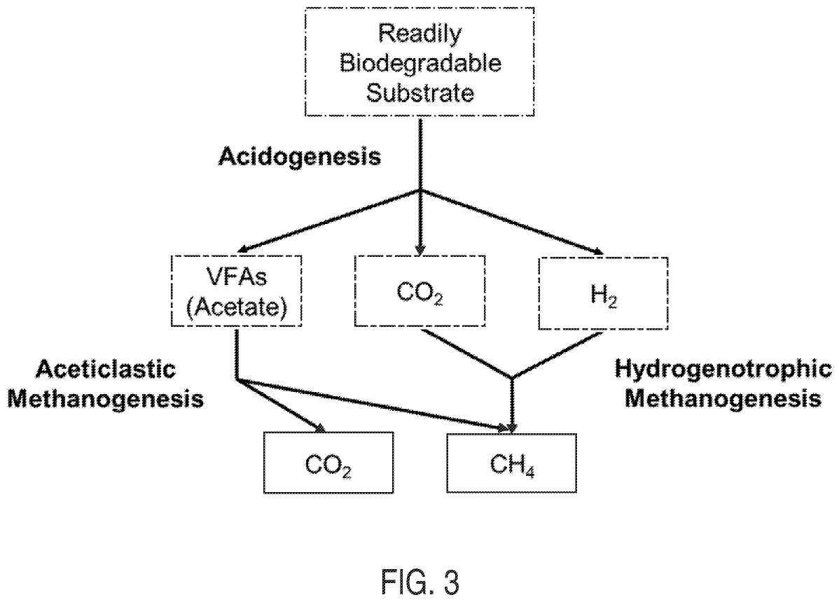

In one aspect, a nonlinear, condensed AD model (SADM in FIG. 2) is implemented for predictions during NMPC optimizations and is based on the AM2 model by Bernard et al., (2001) with modifications. The reaction pathways incorporated are depicted in FIG. 3. In some embodiments, a two-step process is provided (acidogenesis and methanogenesis) as per the AM2 model. In some embodiments, the hydrogenotrophic methanogenesis pathway is included as an addition to the list of possible pathways for methanogenesis. Considering only the aceticlastic methanogenesis pathway (as assumed in AM2) may underestimate overall production of CH₄ and also contribute to COD balance errors. The list of assumptions for the SADM may include:
1. Two step process for the overall degradation of readily biodegradable organic substrate to CH₄: acidogenesis and methanogenesis
2. Instantaneous degradation of organic substrate to VFAs
3. VFA intermediate products are primarily composed of acetate
4. CH₄ production occurs through aceticlastic and hydrogenotrophic methanogenesis pathways
5. H₂ produced from organic substrate acidogenesis is consumed rapidly or instantaneously in the hydrogenotrophic methanogenesis reaction
6. Total alkalinity (Z) is contributed from acetate anion (intermediate alkalinity), bicarbonate (partial alkalinity), and hydroxide ions alone.

The resulting SADM is a semi-mechanistic model and is composed of the set of Eqs. (1)-(10) for well-mixed AD systems with solids retention (e.g. high rate AD reactor configurations with recycles and continuously stirred tank reactors (CSTRs) with solids retention):

Ordinary Differential Equations (ODEs)

$$\begin{matrix} \frac{{dS}_{a c}}{dt} = D ( {S}_{a c , inf} - {S}_{ a c} ) + {v}_{ a c / S} {r}_{{S}_{S}} ( t ) - {r}_{a c , m} & ( 1 ) \\ \frac{{dS}_{IC}}{dt} = D ( {S}_{IC , inf} - {S}_{IC} ) + {v}_{{CO}_{2} / S} \cdot {r}_{{S}_{S}} ( t ) + {v}_{{CO}_{2} / a c} {r}_{a c , m} - {v}_{{CO}_{2} / {H}_{2}} \cdot {r}_{{H}_{2} , m} - {k}_{L} a ( {S}_{{CO}_{2} \cdot aq} - {K}_{H , {CO}_{2}} {P}_{{CO}_{2}} ( t ) ) & ( 2 ) \\ \frac{dZ}{dt} = D ( {Z}_{inf} - Z ) & ( 3 ) \\ \frac{{dX}_{a c}}{dt} = - \frac{1}{SRT} {X}_{ a c} + ( \frac{{r}_{a c , m}}{{X}_{a c}} \cdot {Y}_{{X}_{a c} / a c} - {k}_{d} ) {X}_{a c} & ( 4 ) \\ D = \frac{{Q}_{S} + {Q}_{{H}_{2} O} + {Q}_{alk}}{{V}_{reactor}} & ( 5 )\end{matrix}$$

Total Methane Production Rate

$${r}_{C {H}_{4}} = {v}_{C {H}_{4} / a c} \cdot {r}_{a c , m} \cdot {M}_{COD , a c} + {v}_{C {H}_{4} / {H}_{2}} \cdot {r}_{{H}_{2} , m} \cdot {M}_{COD , {H}_{2}} \qquad ( 6 )$$

Uptake Rates

$$\begin{matrix}{r}_{{S}_{S}} ( t ) = D ( {S}_{s , inf} - {S}_{S} ( t ) ) & ( 7 ) \\ {r}_{a c , m} = \frac{{q}_{m} {S}_{a c}}{{K}_{S} + {S}_{a c}} \cdot {I}_{p H} \cdot {X}_{a c} & ( 8 ) \\ {r}_{{H}_{2} , m} = {v}_{{H}_{2} / S} \cdot {r}_{{S}_{S}} ( t ) & ( 9 )\end{matrix}$$

### Ionic Speciation and pH

Eq. (10) is solved using the SADM state variables (Z, S_{ac}, and S_{IC}) together with expressions for ionic speciation equilibrium for S_{ac} and S_{IC} to find pH and corresponding ionic species concentrations:

$$Z = [ {Ac}^{-} ] + [ {HCO}_{3}^{-} ] + [ {OH}^{-} ] \qquad ( 10 )$$

The notations for the different variables and parameters in Eqs. (1)-(10) are listed in the nomenclature section. By considering assumption 2, the organic substrate utilization rate in Eq. (7) is simply equal to the net accumulation of the organic substrate by convective transport (linear expression). Also, assuming rapid consumption of H2 during hydrogenotrophic methanogenesis reaction (assumption 5), the rate of hydrogen consumption via methanogenesis in Eq. (9) is correlated directly with the organic substrate utilization rate (Eq. (7)). For aceticlastic methanogenesis, the acetate uptake rate kinetics model in Eq. (8) incorporating pH inhibition is adopted from ADM1 (Batstone et al., 2002) but without the inorganic nitrogen limitation and ammonia inhibition terms. For estimation of liquid to gas transfer rate of the inorganic carbon state (last term in Eq. (2)), the gas phase partial pressure of CO₂ (P_{CO}_{₂}) is taken from the plant output measurements for biogas. With the SADM structure and pathways, the total CH₄ production rate estimation defined in Eq. (6) is more accurate than the estimation in the AM2 model of Bernard et al. (2001). In some embodiments, the control structure involves three MVs (Q_{S}, Q_{H}_{₂}_{O}, and Qₐₗₖ), and the final dilution rate (D) after mixing of these input streams is given by Eq. (5).

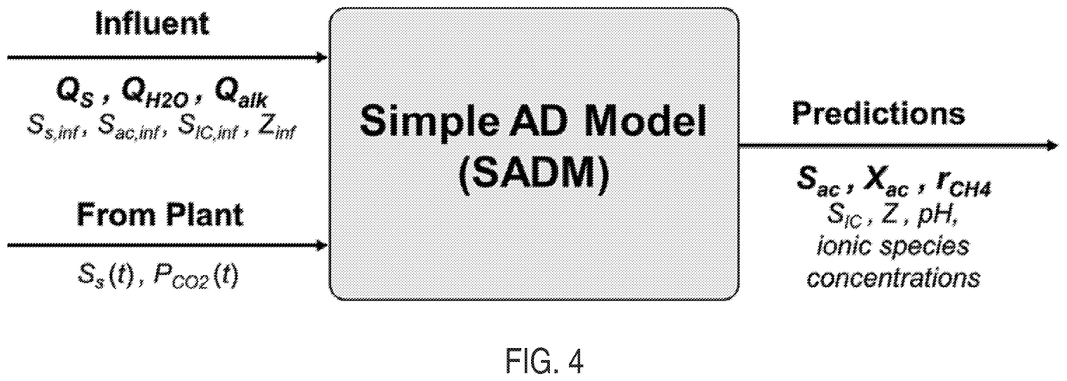

As shown in FIG. 4, in some embodiments, the SADM structure can be interfaced with available information from an AD system or plant to provide predictions of CVs and other relevant variables for the NMPC scheme. The kinetic parameters (stoichiometric coefficients, maximum specific rate of acetate uptake kinetics, half-saturation constant for acetate uptake kinetics, and microbial biomass yields) in the SADM can be identified real time or updated time to time from the off-line measurements (e.g. volatile suspended solids, activity tests, COD of influent and effluent streams). The SADM structure hence provides a practical usability by plant engineers by using measurements available in real AD plants to estimate the main model variables and rates, such that the number of model states and complexity remain small and simple.

The biochemical processes considered for the simple AD model (SADM) are applicable for any suitable process involving AD of readily biodegradable substrates. Some of the terms in the example ordinary differential equations (ODEs) described above may be dependent on particular design and physical characteristics of the reactor system being implemented. In accordance with at least one embodiment, the NMPC scheme involves a high rate, continuous AD system (e.g., an upflow anaerobic sludge bed reactor). Such high rate AD reactor configurations are typically characterized by biomass/solids retention. Hence, the example SADM ODEs described above may be particularly effective for ‘continuous’ AD reactor systems with ‘solids retention’.

As will be apparent to one of skill in the art, when considering models for a real-time NMPC scheme, a lower number of model state variables can provide advantages including efficiency (e.g., computational) and practical effectiveness. The example SADM described herein includes just four such state variables, namely S_{ac}(M), X_{ac}(M), S_{IC}(M) and Z, an improvement over previous models (which, for example, had 6, 13, 35 and more states). As recognized by the inventors, this particular subset of possible model state variables are sufficient for describing AD of readily biodegradable substrates in a continuous AD reactor system with solids retention. In accordance with at least one embodiment, keeping the number of state variables low involves interfacing with an operating AD plant/system to use the available measurements/updates for estimation of model variables and rates.

### NMPC Objective Function

In one aspect, the overall NPMC objective function consists of set-point error tracking terms for the control variables (concentration of effluent volatile fatty acids as acetate, concentration of aceticlastic methanogens in reactor, and total methane production rate) and a cost term penalizing the amount of alkali added (proportional to the volumetric flow rate of the alkali input which is one of the manipulated variables). Also, a number of constraints have been considered for minimization of the NMPC objective function. As an example, for one of the NMPC designs considered (denoted as NMPC Base), three control variables (X_{ac}^{sp}, S_{ac}^{sp}, r_{CH}_{₄}^{sp}) are considered and the overall control objective with constraints is given by Eqs. (11)-(17):

$$\begin{matrix}{min}_{{Q}_{k}} {J}_{P} = {w}_{1} {\sum }_{k = 1}^{k = P} \vert \frac{( {\tilde{S}}_{a c} - {S}_{a c}^{sp} ) \cdot {M}_{COD , a c}}{{S}_{S , inf} \cdot {M}_{COD , S} - {S}_{ a c}^{sp} \cdot {M}_{COD , a c}} \vert + {w}_{2} {\sum }_{k = 1}^{k = P} \vert \frac{{X}_{a c , k}^{sp} - {\tilde{X}}_{a c , k}}{{X}_{a c , k}^{sp}} \vert + {w}_{3} {\sum }_{k = 1}^{k = P} \vert \frac{{r}_{CH 4 , k}^{sp} - {\tilde{r}}_{CH 4 , k}}{{r}_{CH 4 , k}^{sp}} \vert + {w}_{4} {\sum }_{k = 0}^{k = P - 1} \frac{{Q}_{alk , k} \cdot {C}_{alk}}{{Q}_{S , k} \cdot {S}_{S , inf} \cdot {M}_{COD , S}} & ( 11 ) \\ subject to & \\ \dot{X} = f ( t , X , {Q}_{k} ) & ( 12 ) \\ sum \{ {Q}_{k} \} \geq 1000 * {v}_{up}^{m i n} * {A}_{reactor} & ( 13 ) \\ sum \{ {Q}_{k} \} \leq 1000 * {v}_{up}^{m ax} * {A}_{reactor} & ( 14 ) \\ \frac{{\tilde{S}}_{a c , k}}{{\tilde{Z}}_{k}} \leq 0.3 & ( 15 ) \\ \frac{{S}_{S , inf} \cdot {Q}_{S , k}}{{V}_{reactor}} \cdot {M}_{COD , s} \leq {OLR}_{m ax} & ( 16 ) \\ {Q}_{k} \geq {Q}_{LB} \\ where , \\ {Q}_{k} = [ \begin{matrix}{Q}_{S , k} \\ {Q}_{{H}_{2} O , k} \\ {Q}_{alk , k}\end{matrix} ] \\ \dot{X} = [ \begin{matrix}{\dot{S}}_{a c} \\ \dot{{S}_{IC}} \\ \dot{Z} \\ {\dot{X}}_{a c}\end{matrix} ] and X = [ \begin{matrix}{S}_{a c} \\ {S}_{IC} \\ Z \\ {X}_{a c}\end{matrix} ] & ( 17 ) \\ t = k * {T}_{S} & ( 18 )\end{matrix}$$

In another example (denoted as NMPC No rCH4), the methane production rate CV is not considered (i.e. only 2 CVs are targeted) and the resulting NMPC objective function is given by Eq. (19) with the constraints being the same as those defined above (Eqs. (12)-(17)).

$${min}_{{Q}_{k}} {J}_{P} = {w}_{1} {\sum }_{k = 1}^{k = P} \vert \frac{( {\tilde{S}}_{a c} - {S}_{a c}^{sp} ) \cdot {M}_{COD , a c}}{{S}_{S , inf} \cdot {M}_{COD , S} - {S}_{a c}^{sp} \cdot {M}_{COD , a c}} \vert + {w}_{2} {\sum }_{k = 1}^{k = P} \vert \frac{{X}_{a c , k}^{sp} - {\tilde{X}}_{a c . k}}{{X}_{a c , k}^{sp}} \vert + {w}_{4} {\sum }_{k = 0}^{k = P - 1} \frac{{Q}_{alk , k} \cdot {C}_{alk}}{{Q}_{S , k} \cdot {S}_{S , inf} \cdot {M}_{COD , S}} \qquad ( 19 )$$

The proposed objective function (JP) for Eqs. (11) and (19) is optimized over the prediction horizon (P) and the number of unknowns or decision variables for the optimization is equal to the product of the number of MV variables and the control horizon (M).

The constraint in Eq. (12) is the classical optimal control constraint where the optimization has to follow the process dynamics, modelled currently with the proposed SADM. The constraints in Eqs. (13)-(14) place limits on the total influent volumetric flows, ensuring that the total influent volumetric flow is at least being utilized at the minimum design flow but not exceeding the maximum threshold design flow (to avoid washout of biomass). These constraints are specifically applicable for high rate AD systems (case scenario considered in the current work), where upflow liquid velocity (vᵤₚ) is an important design parameter since it affects the hydraulic flow rate and plays an important role in granulation (de Lemos Chernicharo, 2007). To address acid accumulation problems during start-up, the stability constraint given by Eq. (15) is defined to maintain stability indicators within safe limits. In some embodiments, the intermediate alkalinity to total alkalinity ratio is a stability diagnostic indicator and can be below a certain value (e.g., 0.3) to ensure non-inhibitory (towards the AD micro-organisms, particularly methanogens) or neutral pH levels inside the reactor. To avoid organic overloading and to operate the AD process within the practical ranges of OLR, a maximum allowable OLR restriction (Eq. (16)) is considered. Finally, Eq. (17) places lower bound on the individual influent volumetric flows (MVs).

AD Start-Up Performance with the Proposed NMPC Designs

The list of implemented process conditions, NMPC tuning parameters, CV set-points, and other information for testing the proposed control scheme are shown in Table 1. The tuning parameters (M and P), considered as the base settings, were arbitrarily chosen but determined during preliminary simulations to achieve satisfactory performance. In general, the values of CV set-points to use depend on the desired performance and objectives of the implemented process. In some embodiments, the set-points listed in Table 1 were determined from preliminary simulations at steady state using ADM1, with organic substrate feeding at constant feed rate (OLR=15 kg/m³·d) and sufficient alkali addition. The value of maximum permissible OLR listed in Table 1 was selected within the practical range of OLRs for the high rate AD systems. The weights for the NMPC objective function (w₁, w₂, w₃, w₄) were arbitrarily chosen but with higher importance given to the set-point tracking of X_{ac} and r_{CH}_{₄} CVs (favoring build-up of methanogenic capacity during start-up).

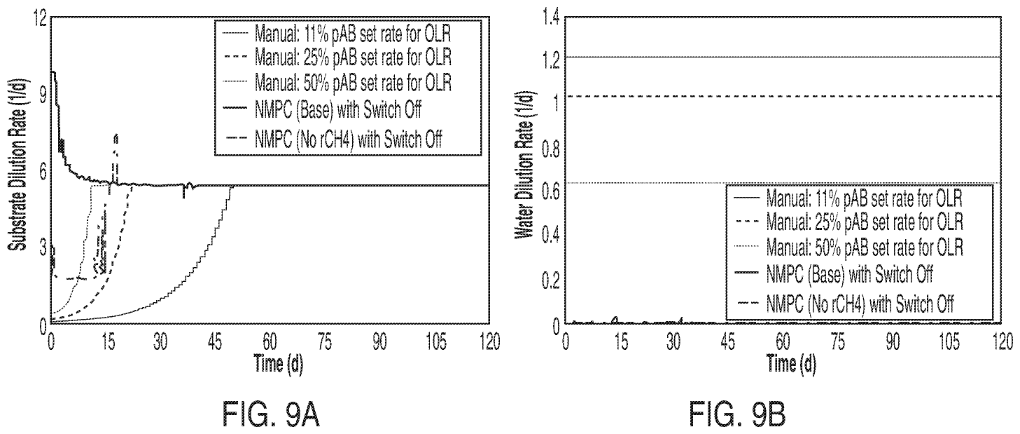

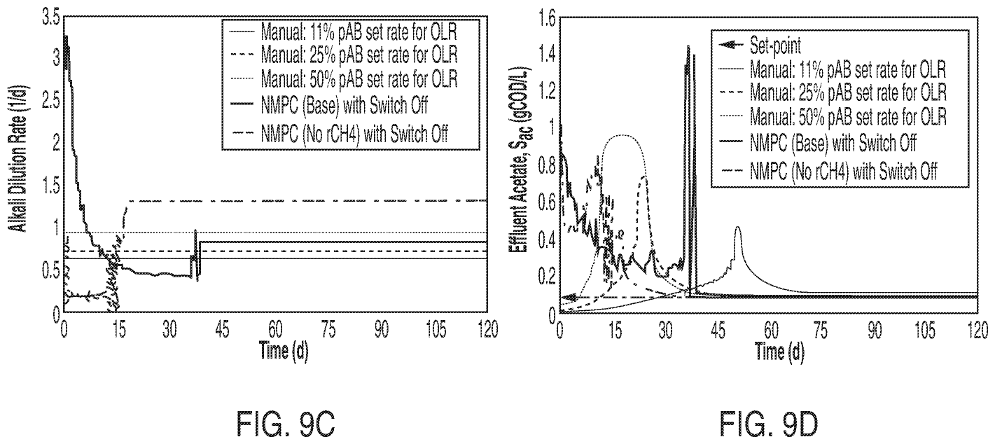

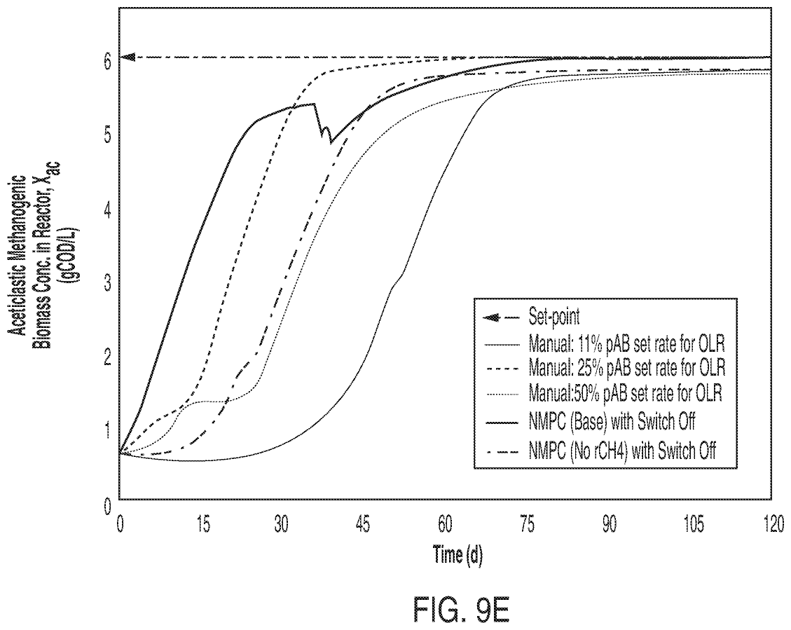

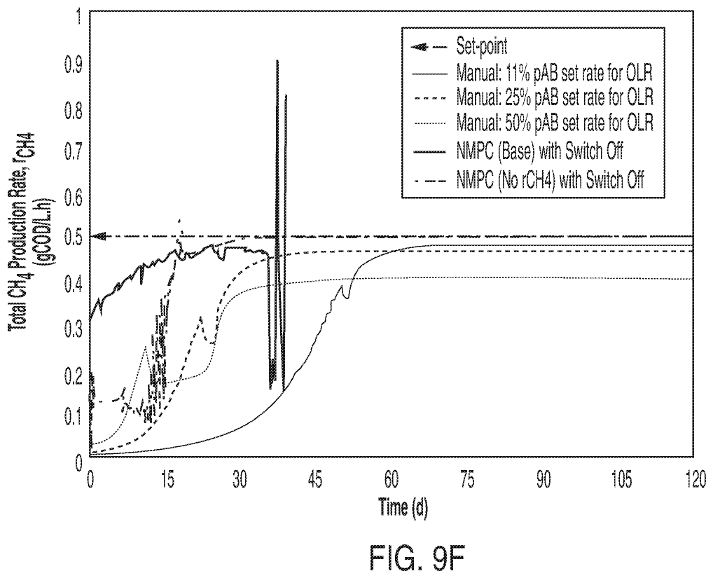

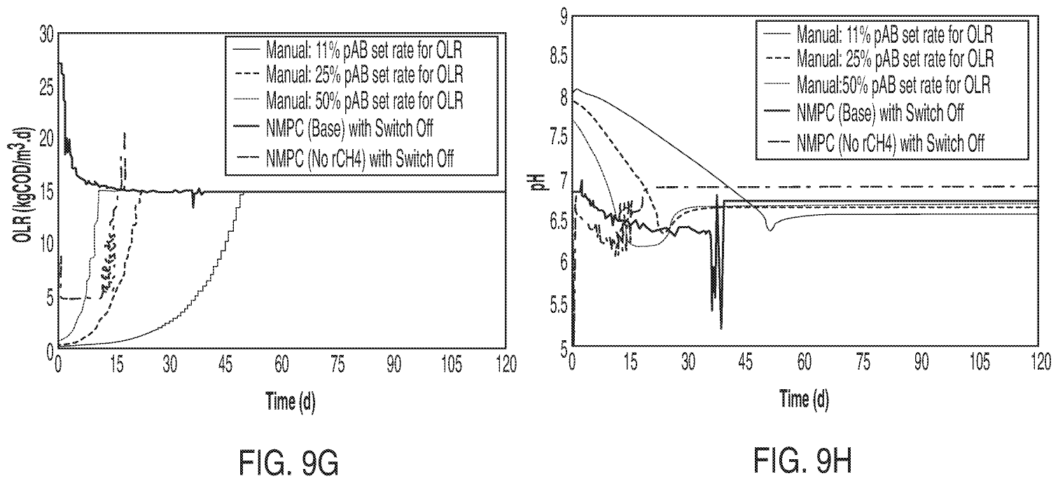

FIG. 9 shows the AD start-up performance and operation with the tested NMPC strategies (NMPC Base and NMPC No rCH4). The NMPC controllers were switched off once the target CH4 production rate (i.e. set-point) was reached (for example, on the 39th day for NMPC_{Base} and on the 18th day for the NMPC_{No rCH4}) and the OLR and the dilution water flow rate were set at 15 kgCOD/m³·d (nominal OLR) and zero respectively. FIG. 9 also includes the process operation and performance with a manual AD start-up management, using the feeding strategy of Angelidaki et al. (2006). In brief, the organic feeding regime of Angelidaki et al. (2006) at start-up consists of progressively increasing the organic feed rate (OLR) which is calculated based on an activated biomass concept modelled via a linear correlation. In this correlation, the daily increase in activated biomass (AB) was predicted and the OLR to set each day was determined as a percentage of the AB (pAB) accumulated till that time. To determine the most appropriate value of pAB to use, three different values were tested (11%, 25% and 50%). The external alkali input and dilution water flows were adjusted for the manual strategy to ensure stable performance and operation within the flow limits. The OLR values as per this manual strategy were fed till achievement of nominal OLR of 15 kgCOD/m³·d).

In general, all the start-up strategies tested (NMPC and manual) yield stable and satisfactory performance by successfully achieving or nearly achieving CV set-points (FIG. 9 (d)-(f)) without any process destabilizations or failure in the long run (FIG. 9 (d)-(f) and (h)) according to model predictions. During start-up, however, significant differences were evident in terms of the times required to reach near the CVs set-points, the organic substrate feeding paths followed, and the process performance profiles during the initial periods. The manual strategy yields sluggish responses in CVs overall (FIG. 9 (d)-(f)) due to the slow rate of OLR increase. The percentage of AB for the manual strategy (i.e. the method of Angelidaki et al. (2006)) could be increased for a faster OLR operation. Simulations with higher percentages of pAB (25% and 50%) yielded faster responses in OLR and CVs but at the cost of lower methane production rates at steady state and higher extents of VFA (as acetate) accumulation during start-up phase.

NMPC variations in which CH₄ production is one of the priorities during start-up (i.e. NMPC Base), lead to a start-up strategy with a very aggressive initial feeding at very high OLRs (contrary to conventional practices) as seen in FIGS. 9(a) and (g), but at a high expense of alkali dosing initially (FIG. 9 (c)). In the case where methane production is not prioritised (NMPC No rCH4), the proposed inputs follow low to higher OLR feedings (more in line with conventional start-up practices), under very low requirement of alkali input initially. However, in the long run, the required levels of alkali dosing are different. Both NMPC scenarios lead to fast start-up of the process in comparison to the manual strategy in terms of the CH4 production output. In some embodiments, the simpler NMPC design without targeting the CH4 production CV (i.e. NMPC No rCH4) results in the fastest achievement of CVs toward the set-points and thus can be used for automatic and optimal AD start-up management. With regard to alkali dosing in the long run, the flow of alkali input after switching off the NMPC controllers could be adjusted (manually or preferably by another control scheme) instead of the flows set in the current work (the scope of current work is limited to operation suggestions within the duration of automatic mode in the start-up period and not the long run).

### Example NMPC Parameters

| | |
|---|---|
| Sampling Time (h) | 12 |
| Control Horizon, M | 4 sampling time steps (2 days) |
| Prediction Horizon, P | 8 sampling time steps (4 days) |
| Objective function weights | |
| w₁, w₂ (both NMPC designs) | 3, 5 respectively |
| w₃ (NMPC Base) | 5 |
| w₃ (NMPC No rCH4) | 0 |
| w₄ (both NMPC designs) | 0.7 gCOD/gNaHCO₃ |

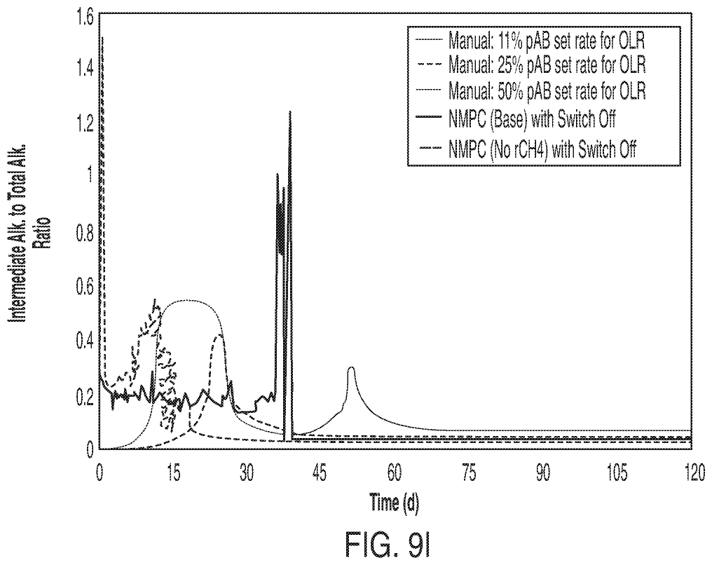

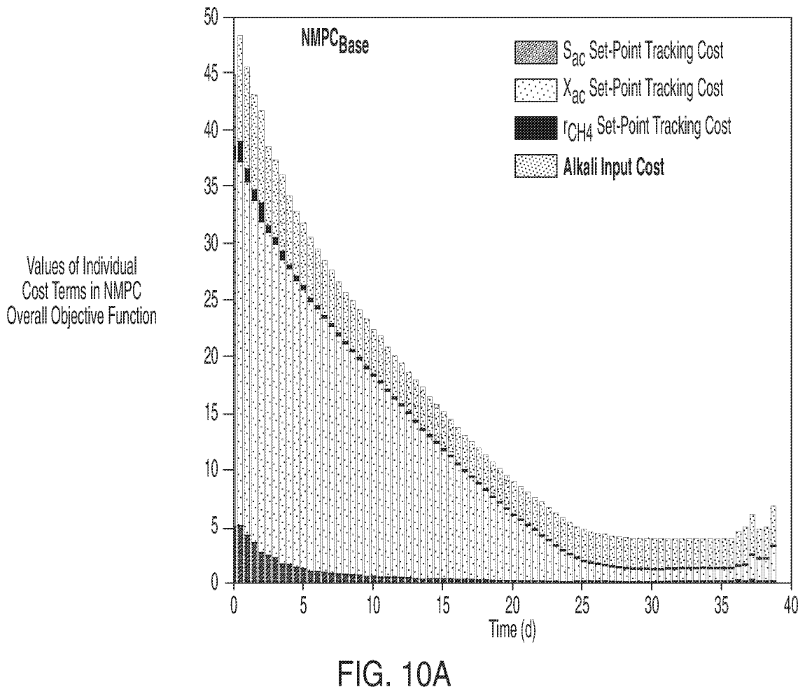

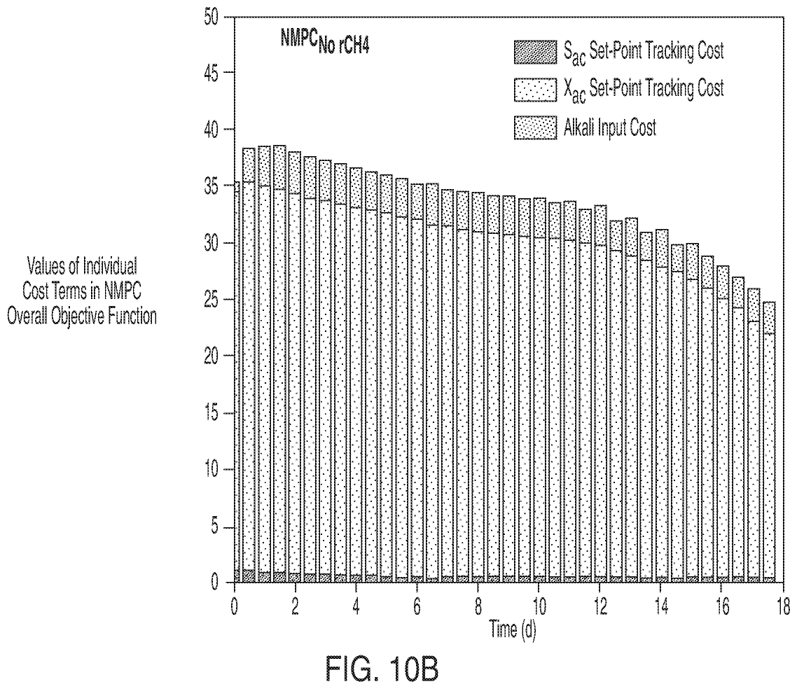

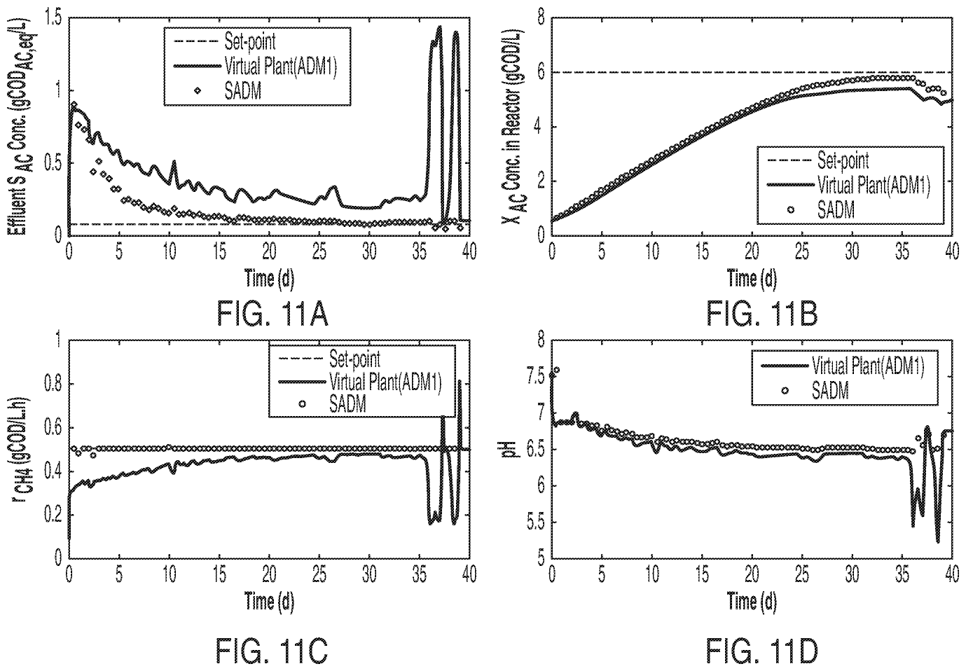

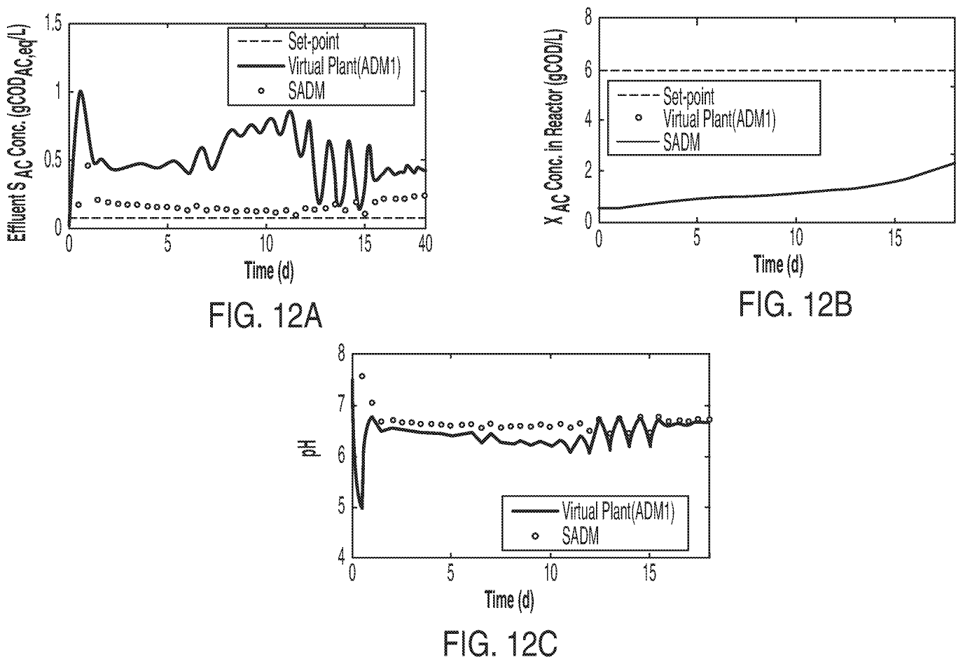

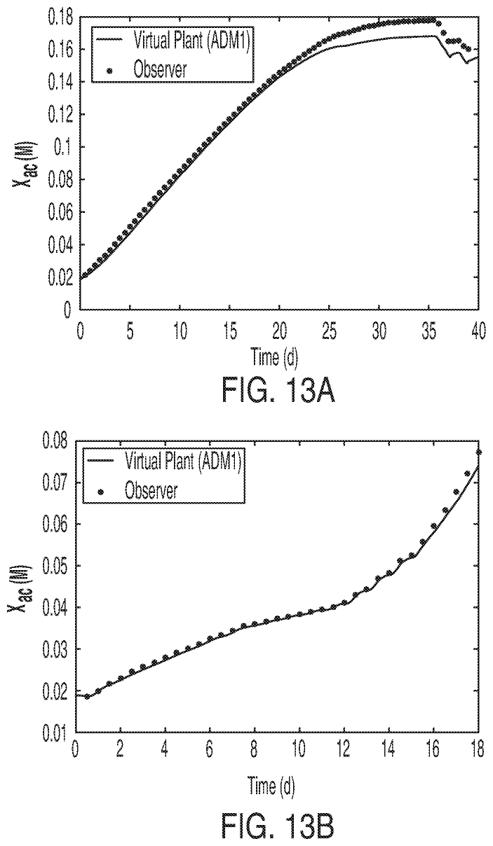

The values of the overall as well as of individual terms contributions to the NMPC objective functions (Eqs. (11) and (19)) after optimisations are shown in FIG. 10. The specifically developed prediction model for the NMPC (sADM) showed excellent agreement with the much complex virtual plant model (ADM1) when looking at the process output predictions of Xac and pH. (see FIGS. 11 and 12). The proposed sADM-based state estimator of Xac also showed good agreement with the virtual plant (ADM1) output even though past and delayed (offline) measurement information is used in the estimations (see FIG. 13). The moderate mismatches on the Sac and the rCH4 between sADM and ADM1 may have caused some of the intermittent oscillations in the process MVs and CVs under the NMPC (FIG. 9).

Model inaccuracy issues can be partly overcome in MPC by implementing output feedback strategies which use an output bias correction (Seborg et al., 2011), where the errors or biases between the latest plant measurements and the (k+1)^{th} sampling time instant model predictions (from previous sampling instant) can be added as corrections to the model predictions when conducting control calculations at the latest sampling instant. The NMPC controllers achieved successful AD performance targets during start-up using the simple prediction model (sADM) considering constraints.

A number of quantitative assessments were conducted for a clearer comparison and additional analyses of the tested startup operation and control strategies. Table 7 summarizes the results obtained using the different quantitative indicators. The ITAE criterion (an integral error criterion used in controller design or tuning) gives an indication of the overall performance along the process operation duration in terms of the time-weighted errors between the process outputs and the set-points. For the steady state offset errors (as percentages) reported in Table 7, a zero or negative value for the Sac CV and a zero or positive value for the Xac or rCH4 CV corresponds to achievement of the corresponding set-point at steady state. In terms of achieving the nominal OLR of 15 kgCOD/m3·d, the manual strategy with 50% pAB set sate for OLR and the NMPC design without the methane production rate CV (NMPC No rCH4) was the fastest. However, the manual strategy with 50% pAB set rate affects the AD performance output of the CVs when looking at the values of integral of the time-weighted absolute error (between set-point and the CV) (ITAE) criterion and the CV offset errors at steady state reported in Table 7. Comparing the ITAE and steady state offset values of the Xac and rCH4 CVs across the tested strategies, the NMPC designs provide a significant advantage over the manual strategies in general.

Referring to FIG. 9 (f), both NMPC scenarios lead to faster start-up of the process in comparison to the manual strategy in terms of the methane production output (circa 39 days and 18 days for NMPC Base and NMPCNorCH4 respectively versus 70-75 days for the manual strategies with constant offset errors at steady state as seen in Table 6) and achievement of nominal OLR of 15 kgCOD/m³·d (FIG. 9(g)). In terms of the fastest achievement of CVs toward the set-points (with minor offset error in Sac at steady state) and the nominal OLR, the simpler NMPC design without targeting the methane production CV (i.e. NMPC No rCH4) appears to be the preferred choice for automatic and optimal AD start-up management. Also, successful onset of the process (stable levels of CVs and pH) is predicted after switching off the NMPC (FIG. 9 (d)-(f) and (h)). Alkali dosing in the long run could be later adjusted manually or by another control scheme. 3.5. Robustness and performance analyses of the NMPC AD start-up under different scenarios.

A number of practical case studies and scenarios were evaluated to assess the two NMPC designs (NMPC Base and NMPCNo rCH4) for robustness and possible improvements. The details on these scenarios and the results obtained have been provided in Appendix E. In brief, the following cases were simulated:

1. Model (sADM) mismatch/bias correction, in which at each sampling event, the differences between the plant outputs and sADM predictions are added as corrections to the sADM predictions for the next NMPC optimisation step.
2. Assumption of online Xac measurement availability without any delays (i.e. no Xac estimator) in order to assess the impact of the state estimator.
3. Instrument error via random noise ranging between −10% and 10% change relative to actual measurements of the four sADM states and CH4 production rate.
4. Measured disturbances of substrate influent COD (2 tests: 75% decrease and 75% increase in the base influent COD of 2.75 gCOD/L at 10th day for 15 days)
5. Unmeasured disturbances of substrate influent COD (4 tests: +5% and +75% random changes in influent COD of 2.75 gCOD/L; 80% decrease and 80% increase in influent COD of 2.75 gCOD/L at 10^{th} day for 15 days)

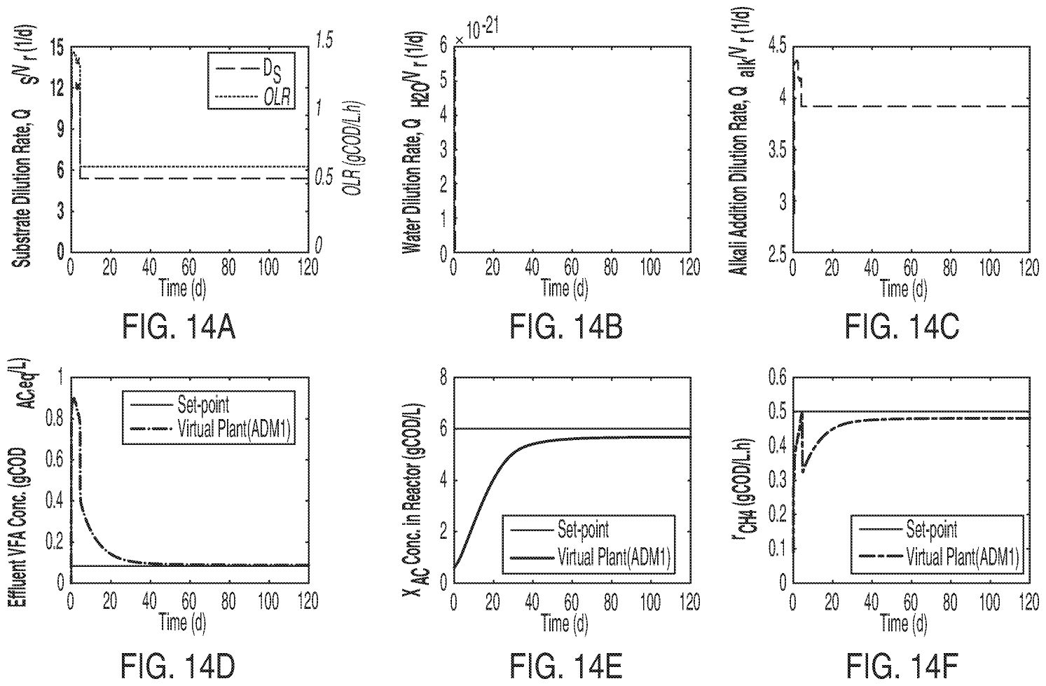

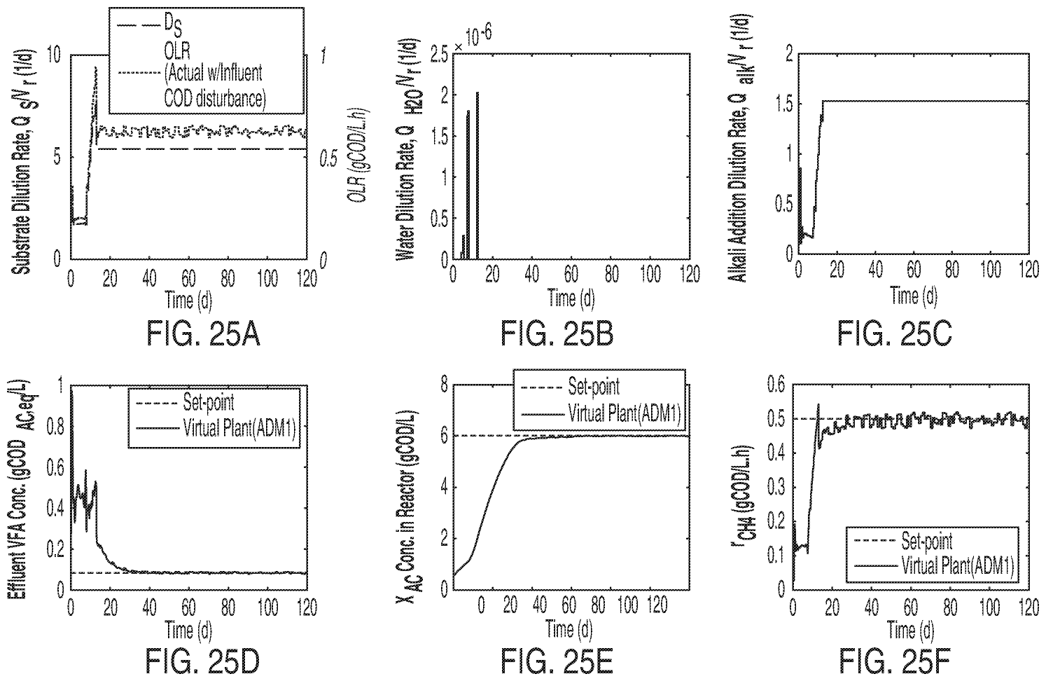

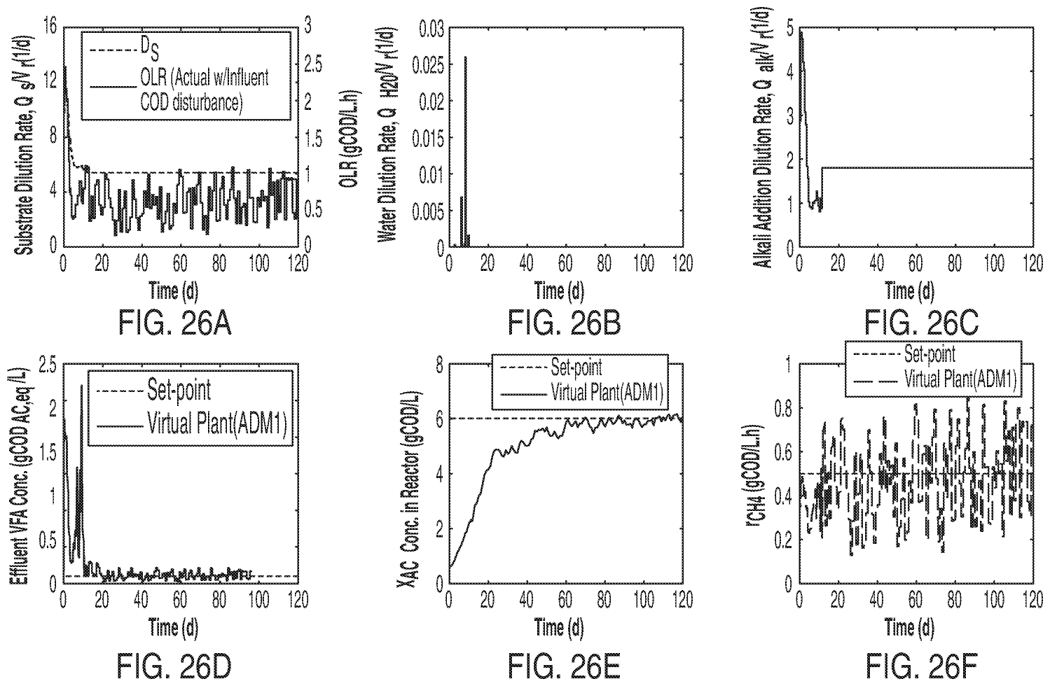

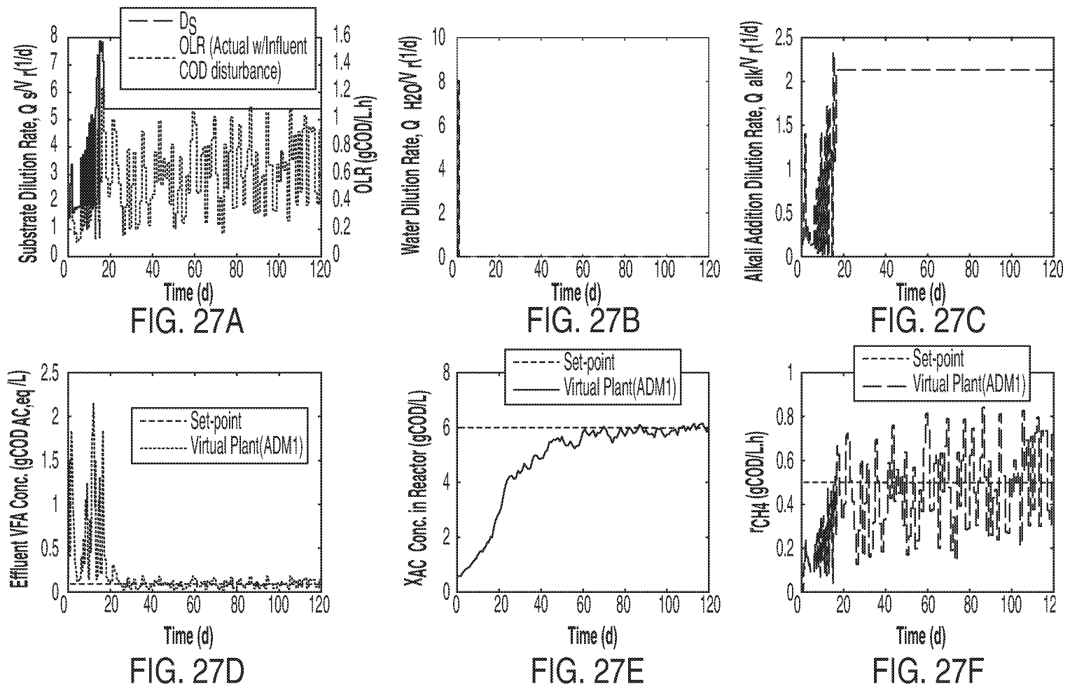

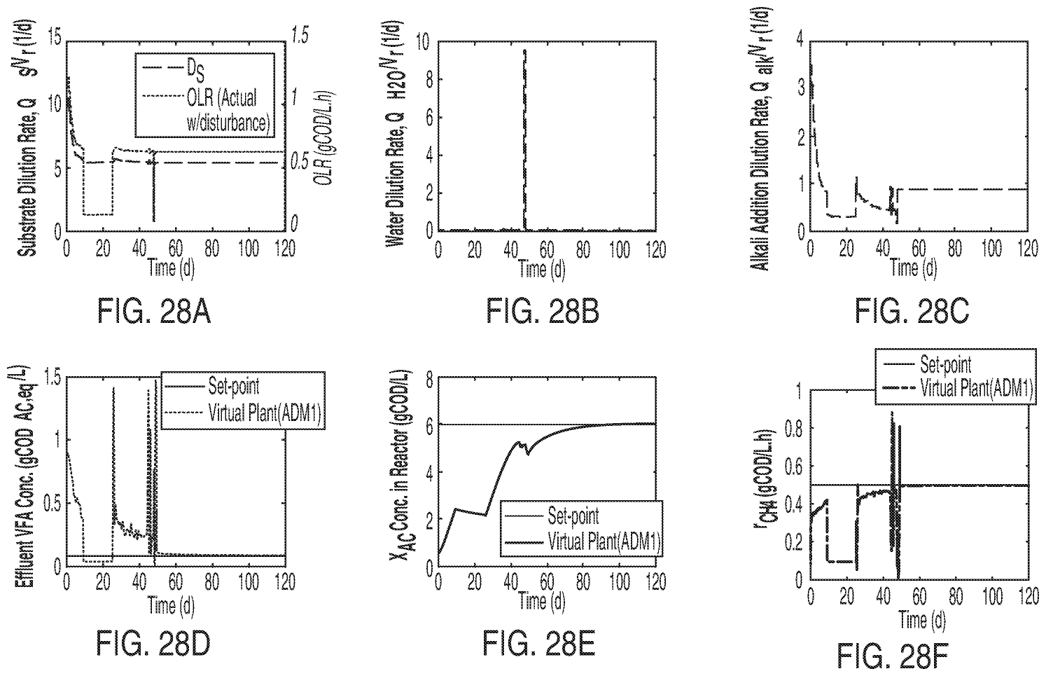

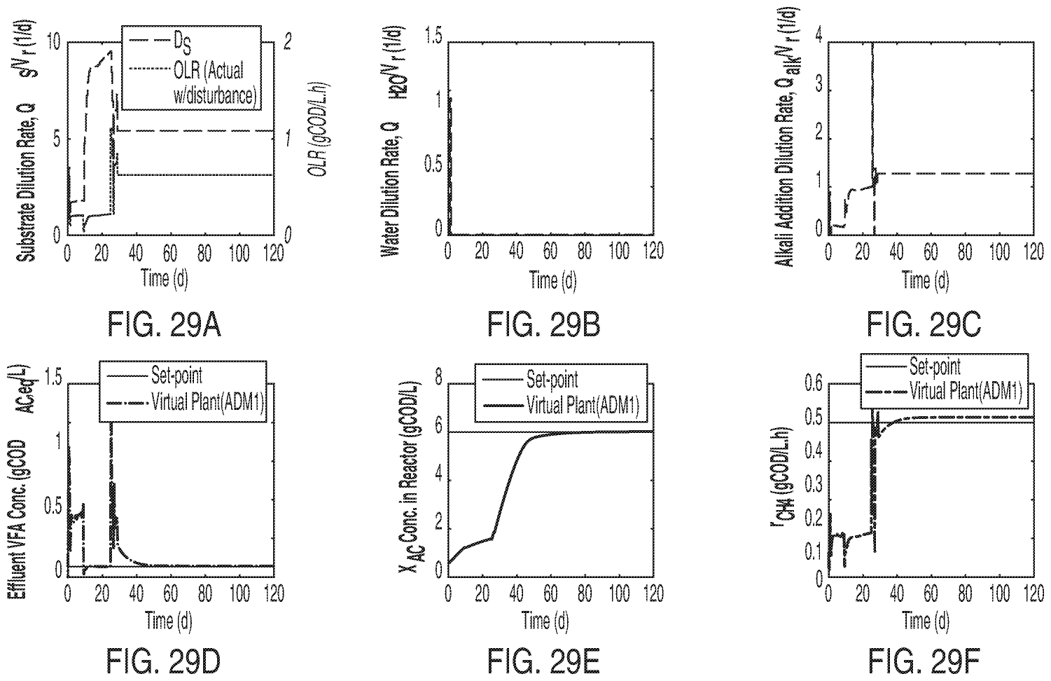

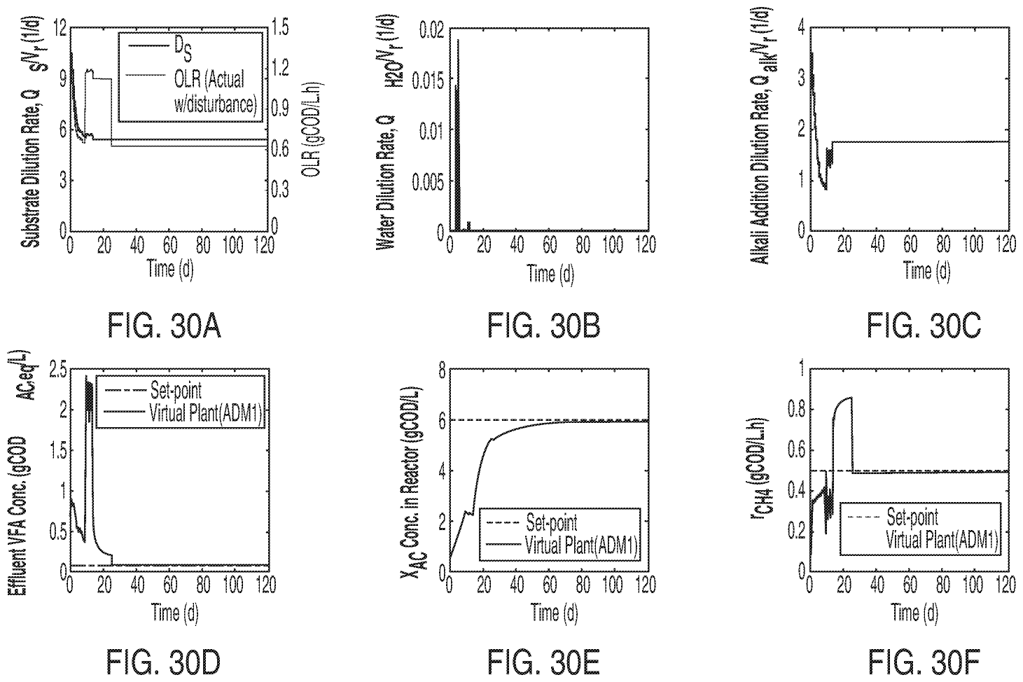

The results (figures and quantitative assessments) for the above cases are provided in Appendix E (Table E and FIGS. 14 to 31).

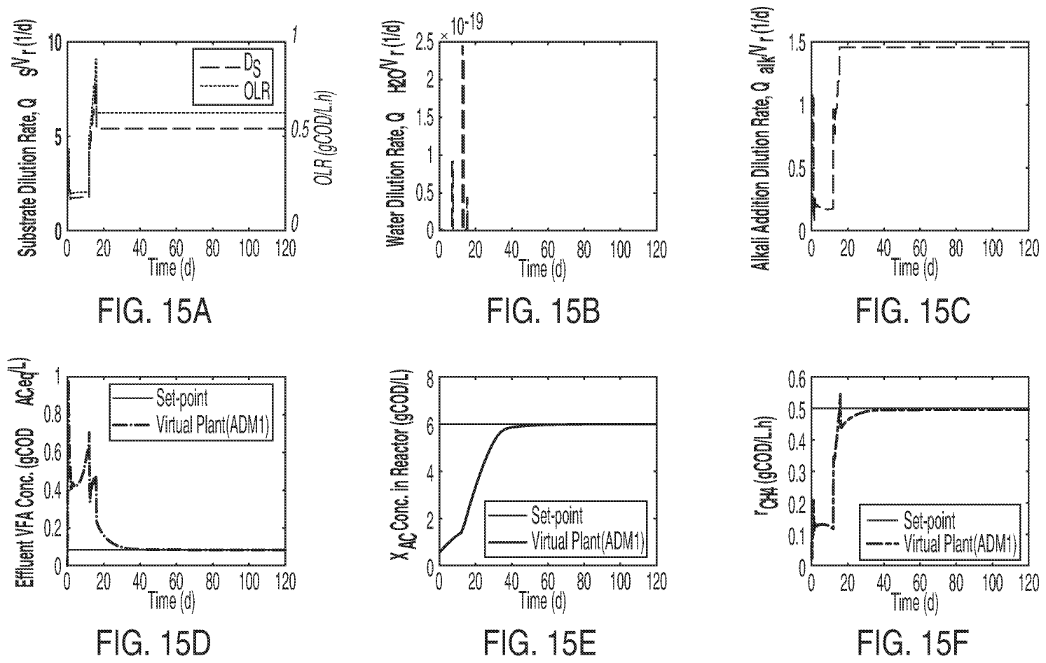

For the scenario involving model bias correction, both NMPC designs yield faster performance with respect to switching to manual operation at constant nominal OLR of 15 kgCOD/m3·d (Table E) compared to the results in Table 6. However, the NMPCBase design yields constant CV offset errors relative to setpoints (Table E and FIGS. 14 (d)-(f)) indicating that the setpoints for the Xac and CH4 production rate could not be reached at steady state. The NMPCNo rCH4 yields a better performance according to the values of ITAE and CV set-point offset errors (Table E and FIGS. 15 (d)-(f)) when compared to the results with NMPC Base.

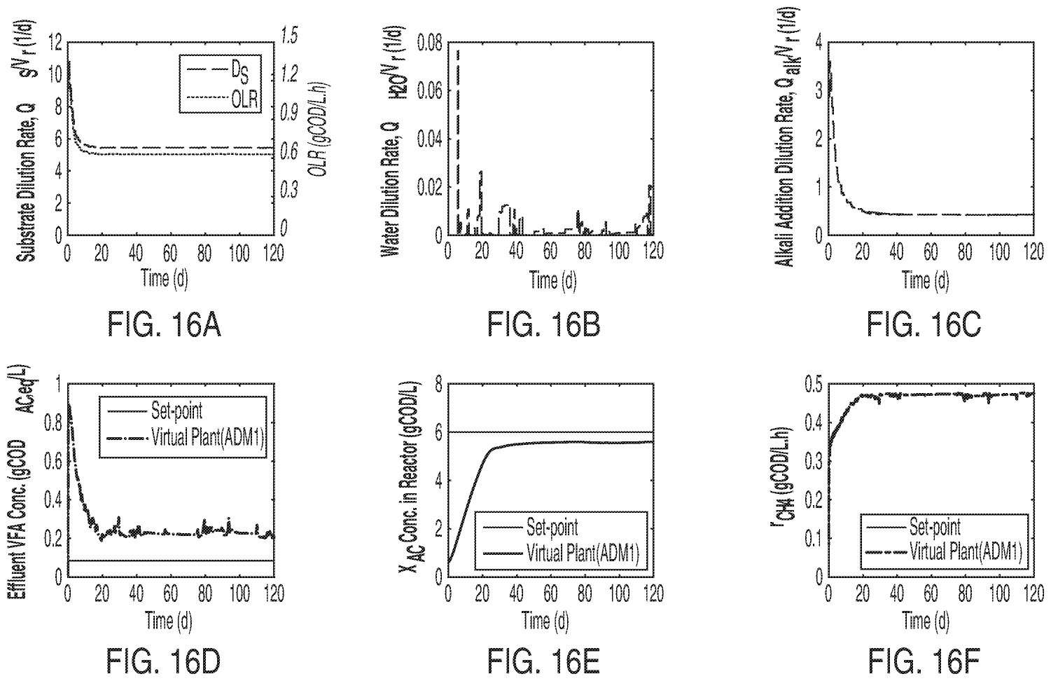

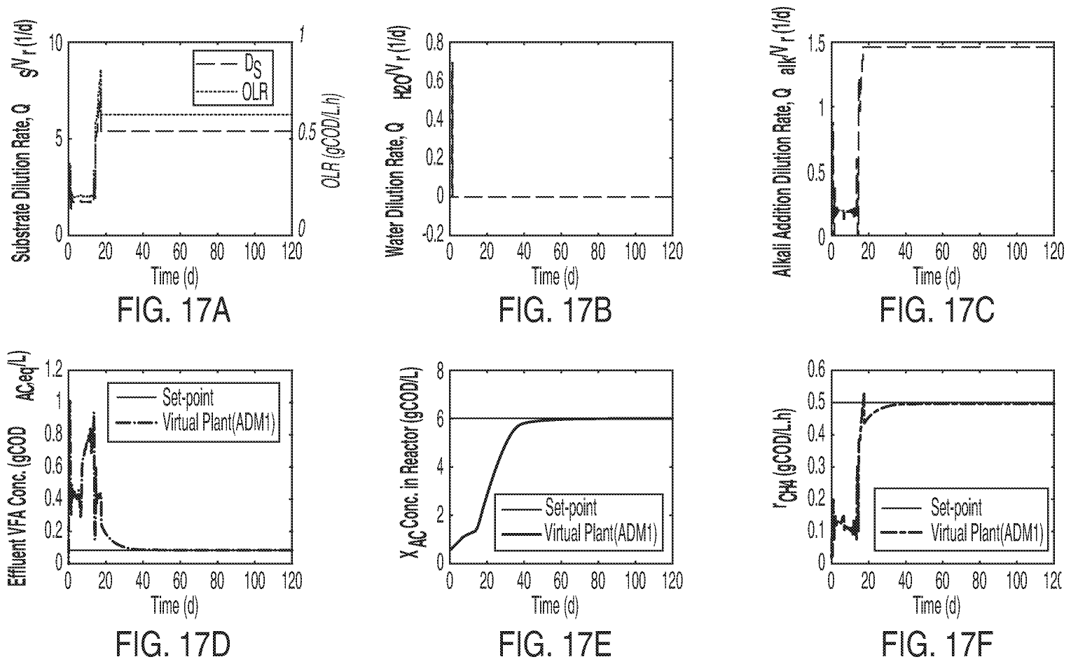

With the assumption of Xac online measurement availability, the NMPCBase design yields stable performance but is unable to drive the process towards the CV set-points (FIG. 16 (d)-(f)) which was unexpected. With the NMPC No rCH4 design, the start-up performance (Table E and FIG. 17) is similar to that obtained under base scenario (Table 6 and FIG. 4). This indicates that the estimator works well without the availability of online measurement for Xac. This is expected due to the previous good agreement between the estimator and the plant output for Xac (FIG. 14).

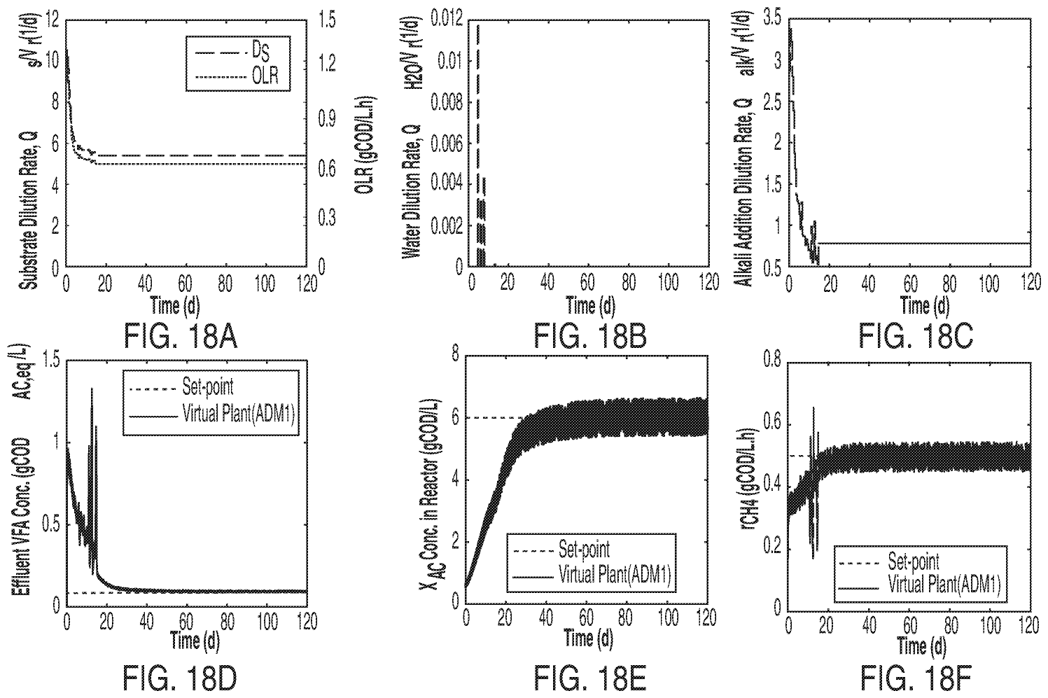

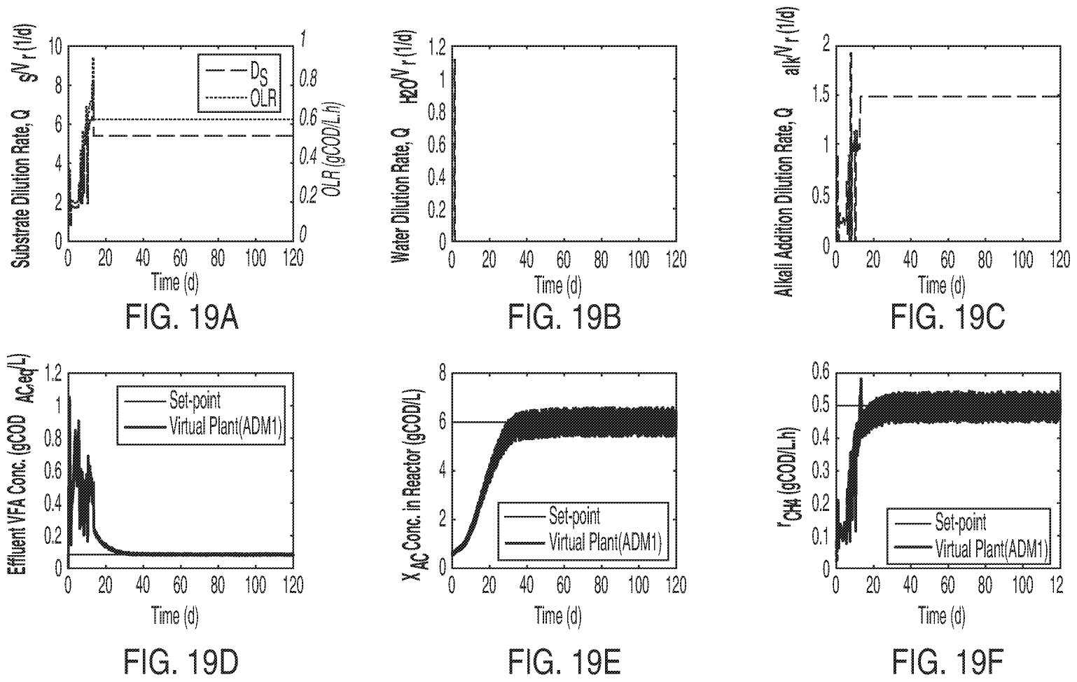

Despite the instrument errors occurring throughout the process operation time, both NMPC designs yield satisfactory results as the process is driven towards the CV set-points without any failures (Table E and FIGS. 18 and 19) and without any oscillations in the optimal set of MVs implemented by the NMPC.

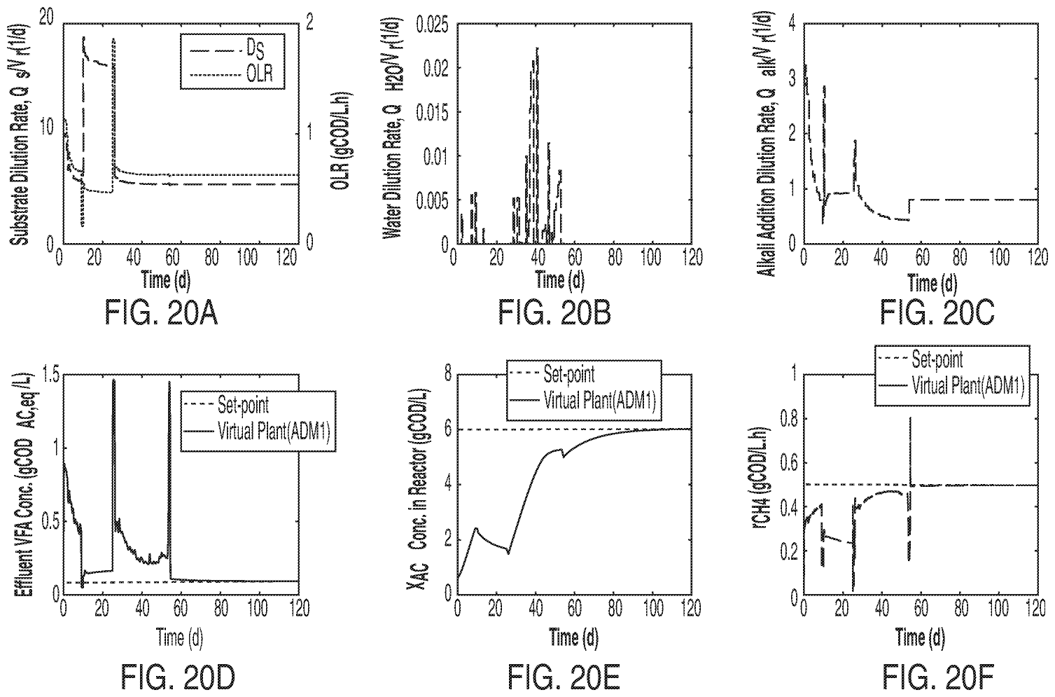

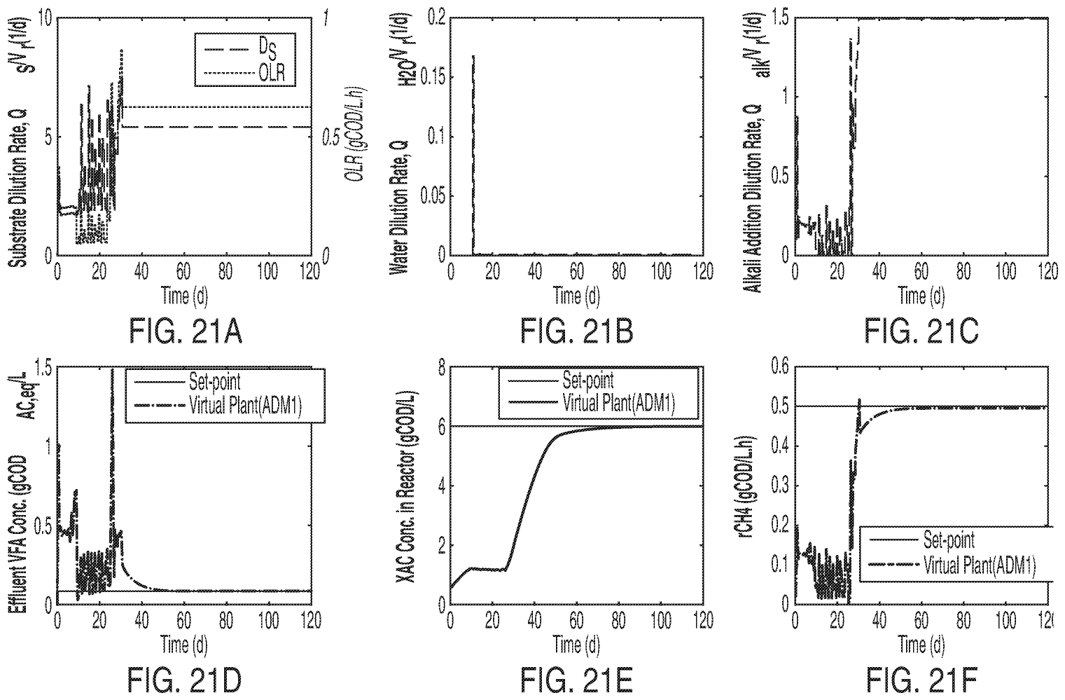

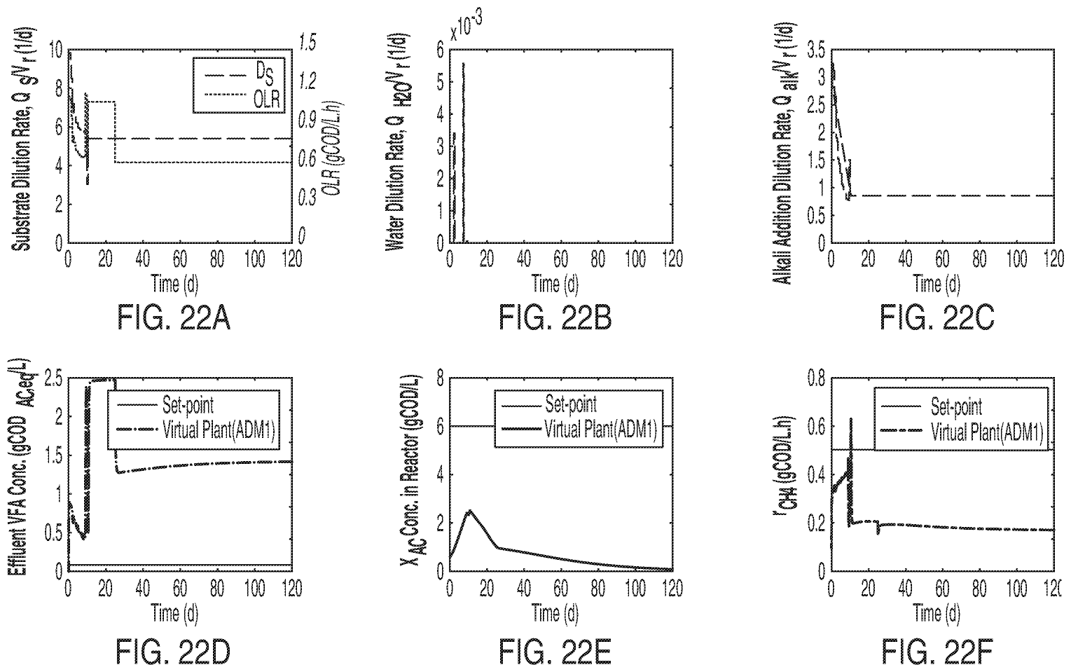

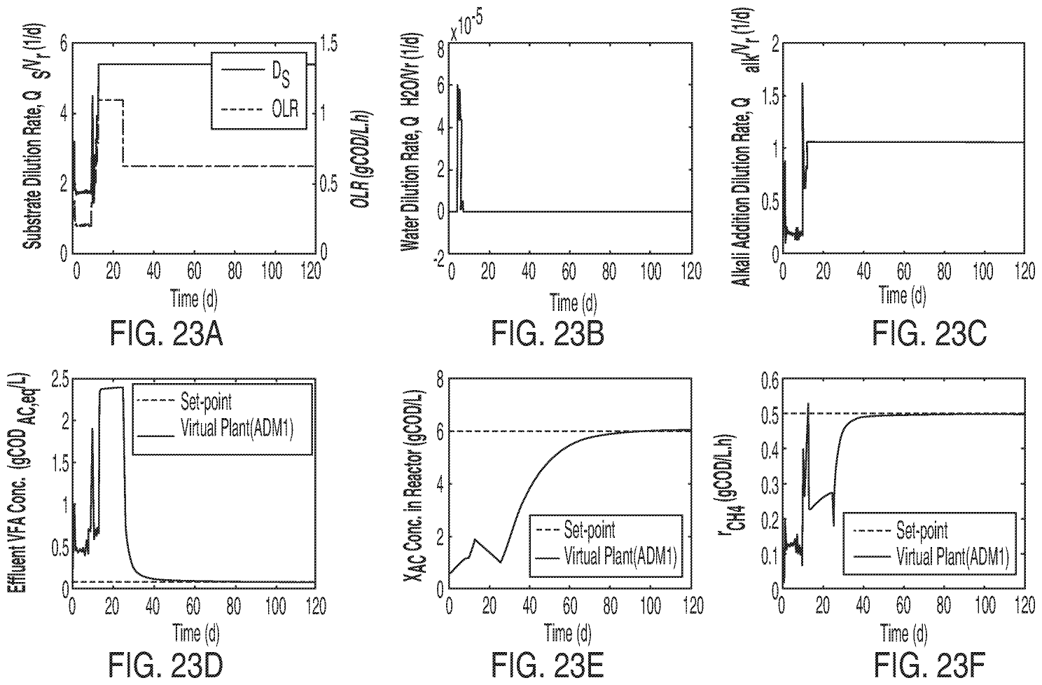

With measured disturbances of the influent COD of the substrate, the NMPCBase design yields stable performance but with significant intermediate variations of CVs (FIG. 20) for the disturbance involving reduction in influent COD. However, when subjected to disturbance involving increase in influent COD, the start-up performance is significantly poor with the NMPCBase design (Table E and FIG. 22 (d)-(f)). Irrespective of the nature of disturbance, the NMPCNo rCH4 yields stable performance while successfully meeting the CV set-points at steady state but with oscillatory behavior and variations during start-up (FIGS. 21 and 23). Also, the time taken to switch to the nominal OLR of 15 kgCOD/m3·d and for conditions to stabilize around the set-points is longer when the disturbance involves reduction in influent COD (Table E).

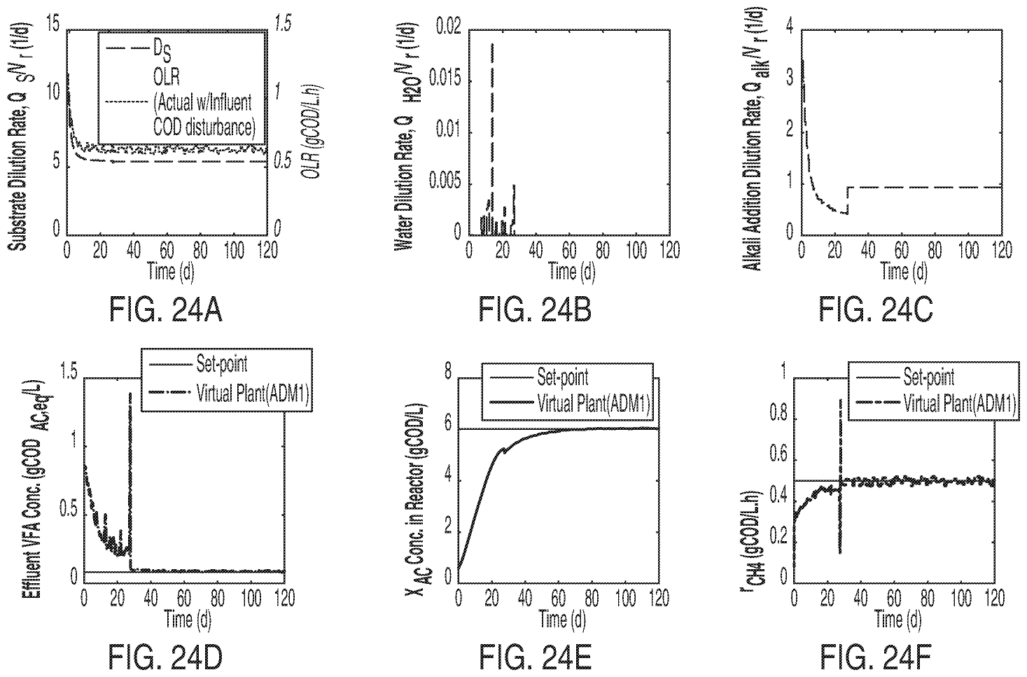

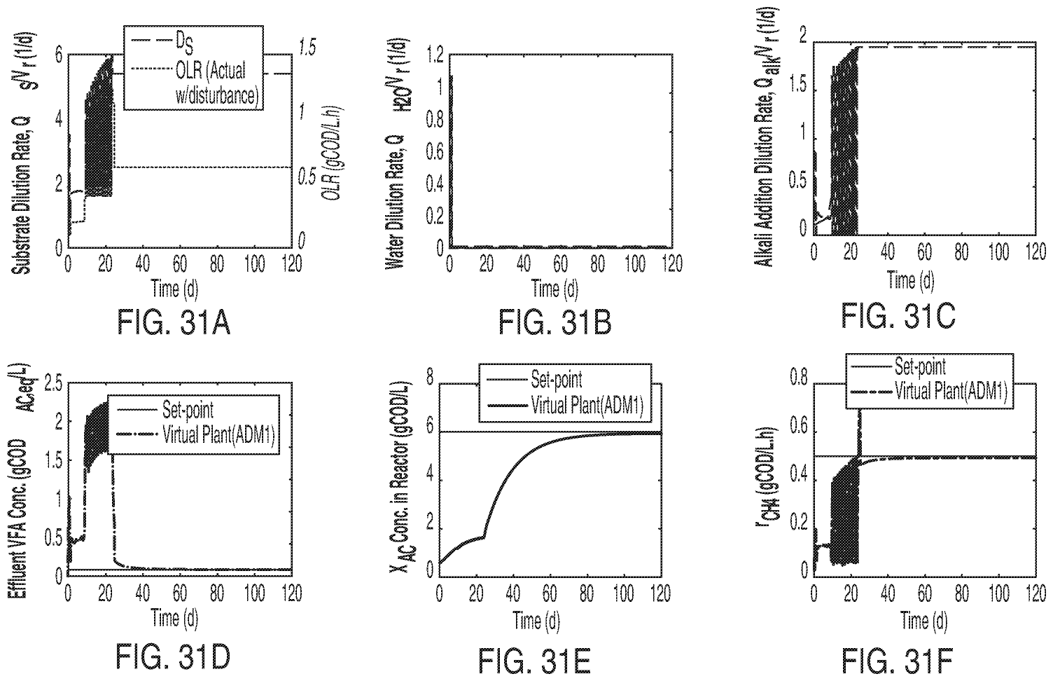

Under unmeasured disturbances in the influent COD (i.e. the sADM is unaware of the changes in substrate influent COD concentration), both NMPC designs are able to drive the process towards the set-points without any stability issues. However, intermediate variations in CVs are observed in general (FIGS. 24 to 31). Also, NMPCNo rCH4 yields highly oscillatory behavior in some cases (FIG. 31).

In summary, the NMPCNo rCH4 configuration appears to provide a more robust and superior performance compared to the NMPCBase alternative configuration against the different scenarios tested. The NMPCBase fails to drive the process towards the set-points for a number of the scenarios considered. This indicates that the NMPC objective function formulation (or in other words, the process variables targeted for optimisation) plays a key role in the level of success of the NMPC scheme for start-up. Also, these results could indicate that the unconventional method of starting with a high OLR at the beginning of operation during start-up (according to NMPCBase design) may not be an effective strategy despite its possible apparent optimality respect to the conventional approach of low to high OLR.

### Virtual Ad Plant Model

For accurate representation of the process plant or system against which the control scheme is being designed and tested virtually, the ADM1 model described in Batstone et al. (2002) can be used to simulate the AD process dynamics. In some embodiments, ADM1 is implemented as the virtual AD plant for evaluating the NMPC scheme. An empirical correlation for solids retention time (SRT) with the liquid upflow velocity (vuₚ) was also incorporated (relevant for high rate AD systems) in both the virtual plant model (ADM1) and the SADM, based on the solids retention formulations of Fedorovich et al. (2003).

### AD Process Monitoring During Automatic Operation

On the practical side, the available measurements for AD process monitoring are not enough to evaluate and assess the state of the process (Jimenez et al., 2015). Although a variety of variables are monitored in commercial scale AD plants, the number of variables measured online is quite limited (pH, temperature, biogas flow rate, biogas composition, biogas yield, and partial pressures) while a majority are measured off-line and less frequently. However, at the level of research developments, advanced and novel sensors have been successfully developed in existing studies for online monitoring of key variables (Jimenez et al., 2015). As such, a number of existing studies have successfully developed online measurement tools for VFA levels, alkalinities (partial and total), COD, and volatile suspended solids (VSS) (Boe & Angelidaki, 2012; Falk et al., 2015; Molina et al., 2009; Morel et al., 2005; Nielsen et al., 2007; Steyer et al., 2002).

Considering typical practical limitations in AD instrumentation, in some embodiments the measurements in the NMPC scheme (including SADM) for direct determination or estimation of process variables and parameters is shown in Table 2. The significant issue is the lack of online estimation of microbial biomass concentrations. Soft sensors or state estimators (e.g. Kalman filter) can be used for slow, delayed offline measurements (e.g. methanogenic biomass activity tests for X_{ac}). In the current work, the SADM equations (Eqs. (1)-(10)) were used (in support of having a semi-mechanistic approach) to estimate the aceticlastic methanogenic biomass concentration at every sampling event. In some instances, for estimation via SADM, the set of initial model states are determined from the last available measurements. Also, a 48 h delay in methanogenic activity measurement update was considered when utilizing measurements as initial points for the SADM state estimation of X_{ac} (FIG. 2). In some embodiments, the selected delay time is based on the practical time durations for the specific methanogenic activity tests according to the study of Cho et al. (2005).

### AD System Start-Up Case Scenario

In some embodiments, a simple case study involving optimal start-up control of an AD system treating readily biodegradable substrates is provided for implementing and virtually testing the NMPC scheme (FIG. 2). In particular, the AD conditions used in the experimental study of Subramanyam and Mishra (2013) have been considered and some of the conditions adopted from this study are listed in Table 3. Since the organic substrate of interest is a readily biodegradable substrate (glucose), modeling this scenario with the SADM is useful. The reactor configuration implemented in the study of Subramanyam and Mishra (2013) is an upflow anaerobic sludge blanket (UASB) reactor, which is a successful and most widely used high rate AD technology (Lier et al., 2008). Due to the high biomass retention in these systems, high OLRs can be achieved (as high as 60 kgCOD/m³·d in case of expanded granular sludge bed and fluidized bed reactors). According to statistics of Van Lier (2008), the application of high rate AD reactors has been potentially growing over the years.

### Simulation of the NMPC Framework

For virtual plant simulation with ADM1 predictions, benchmark ADM1 parameter values reported in Batstone et al. (2002) for mesophilic high rate systems have been used. The parameters in the SADM equations listed in Eqs. (1)-(10) (ν_{ac/s}, ν_{CO}_{₂}_{/S}, ν_{CO}_{₂}_{/ac}, ν_{CO}_{₂}_{/H}_{₂}, ν_{H}_{₂}_{/S}, ν_{CH}_{₄}_{/ac}, ν_{CH}_{₄}_{/H}_{₂}, qₘ, K_{S}, pH inhibition parameters, k_{L}a, k_{d}, Y_{X}_{_{ac}}_{/ac}) were adopted from ADM1. A maximum limit on the concentration of total solids was incorporated in ADM1 using an empirical function to ensure washout of excess solids from the system. For the AD start-up control simulations, the microbial biomass proportions in the solids predicted at steady state from preliminary simulations were considered to mimic the conditions of AD start-up with an adapted inoculum. Moreover, based on the AD start-up and operation guidelines of Hickey et al. (1991) for high rate systems including UASB, an inoculum amount equal to 10% of reactor volume was considered at start-up. Hence, for the initial ADM1 (virtual plant) states, the values of all the state variables except the concentrations of microbial biomass (from preliminary simulations with dilution described earlier) and nutrients (inorganic carbon, inorganic nitrogen, cations, and anions) were set as zero inside the reactor. The initial biomass concentrations for the preliminary simulations were estimated using the VSS and SMA values reported in Table 3.

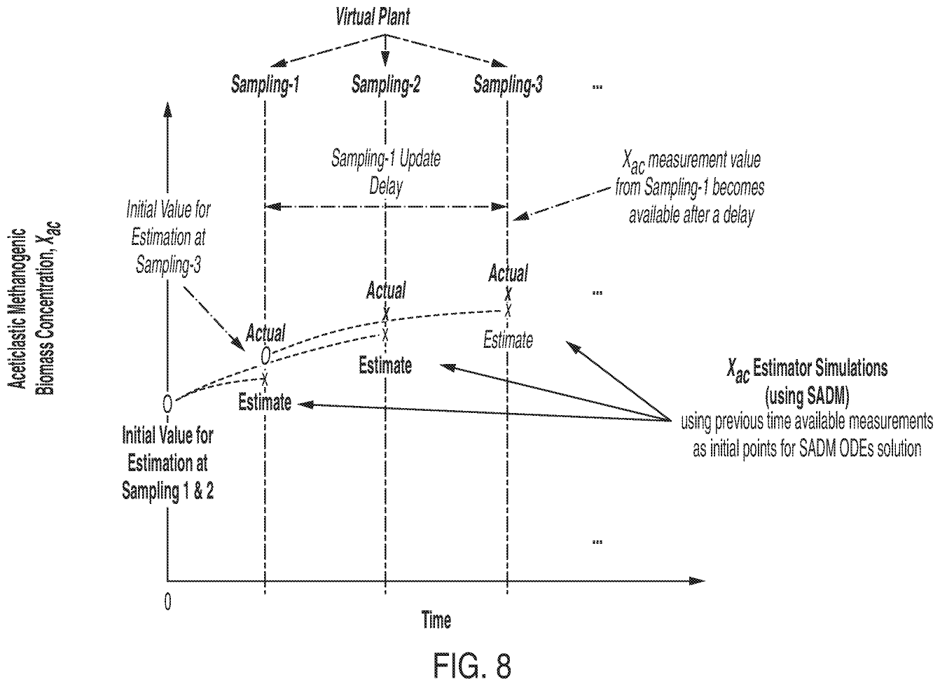

In one aspect, the interfacing of the virtual plant (ADM1) outputs into NMPC and SADM compatible variables is shown in Table 4. The effluent organic substrate concentration (Ss) and CO₂ partial pressure (P_{CO}_{₂}) updates from the virtual plant (ADM1) used for SADM equations are held constant when conducting SADM predictions during NMPC optimization at a given sampling time instant. At the next sampling event, these previous updates are replaced with the latest values and the procedure is repeated. The remaining virtual plant outputs (except the CH₄ production rate) listed in Table 4 are used as updated initial conditions for the SADM state variables. The aceticlastic biomass concentration (X_{ac}) at a given sampling event is estimated using SADM as the state estimator (FIG. 2), utilizing past available measurements as initial points (estimation methodology illustrated in FIG. 8). In case of update for the total alkalinity (Z) state variable of SADM, some of the plant output updates(S_{ac,eq}, S_{IC}, and pH) are used to compute Z as in Eq. (10).

The proposed NMPC scheme was implemented in MATLAB® R2015b. MATLAB function files and a Simulink® file were created for achieving the tasks in the control scheme implementation (FIG. 2). The equations for ADM1 (virtual plant) were programmed and solved using the modified version of the modelling framework of Rodiguez et al. (2009). The SADM ODEs were solved using the ode45 built-in MATLAB solver to get model predictions during NMPC optimizations. Finally, the NMPC optimizations were solved using the fmincon built-in MATLAB solver with the sequential quadratic programming (SQP) algorithm. The simulations were run on an Intel® Core™ i5-3230M 2.60 GHz processor with 8.00 GB RAM & 64-bit Windows 10 operating system.

### AD Process Monitoring

In one aspect, AD process monitoring is accomplished using online measurements, off-line measurements, or both. An extensive review of instrumentation and control in AD processes is provided by Jimenez et al. (2015, Reviews in Environmental Science andBio Technology, Vol. 14, pp. 615-648), which is incorporated by reference herein. In addition, the report by Kock & Eberlein (2012, see the internet at portal.ea-stmk.at/documents/20181/25550/D_5_1_Best_Practice_Monitoring.pdf/13b61dbe-a886-454d-97e4-24aeada73cab) provides a list of variables monitored in some of the existing AD/biogas plants in Europe. Table 5 provides non-limiting examples of conventional variables and parameters and sensors or equipment used to detect or measure the variables or parameters in existing commercial AD systems. The preference of having one specific sensor or measurement technique over another may depend on the equipment availability and monitoring cost constraints of the AD plant being implemented.

Table 6 provides non-limiting examples of online variables and parameters, and sensors or equipment used to measure or detect the variables or parameters, in research facilities. Some of these sensors have been validated in full scale AD systems.

### Anaerobic Digestion Systems

The models described herein can be used in AD start-up processes. The process of AD occurs in several steps and typically involves a community of micro-organisms that interact together in the absence of oxygen to decompose organic matter into biogas as the main end product. Biogas is mostly methane (CH₄) and carbon dioxide (CO₂), with very small amounts of water vapor and other gases. The carbon dioxide and other gases can be removed, leaving only the methane. Biogas is a renewable energy source that can be used in a variety of ways. For example, biogas can be valorised in a combined heat and power (CHP) unit to produce electricity and heat. Alternatively, biogas can be upgraded to biomethane to reach the purity of natural gas and be injected into the municipal gas grid or be used as transportation fuel. The AD process also generates stable residue called “digestate” as the other main end-product. Digestate is a wet mixture that is usually separated into a solid and a liquid (dewatering). In some cases, the digestate can be rich in nutrients and hence, used as fertilizer for crops.

Methanogenic archaea (micro-organisms involved in the terminal bioconversion reaction step of the overall AD process) are considered rate-limiting key-players of the AD process due to their slow growth rates and high sensitivity to different environmental conditions (Weiland, 2010). Thus, process inhibition can often be encountered due to an imbalance between the VFAs and other precursor-producing bacteria and methanogenic archaea (Ahring et al., 1995). Moreover, many environmental parameters such as the digestion temperature (Luo et al., 2015), the organic loading rate (OLR) (Goux et al., 2015) or even the substrate type (Westerholm et al., 2016) greatly influence the microbial community development and structure.

The AD process is typically divided into four main stages (hydrolysis, acidogenesis, acetogenesis and methanogenesis), each involving different microbial communities (Weiland, 2010). Hydrolysis refers to the break down of large, complex polymers like carbohydrates, cellulose, proteins and fats by hydrolytic enzymes into simple sugars, amino acids, and fatty acids. During acidogenesis, simple monomers are broken down into volatile fatty acids. During acetogenesis, the products of acidogenesis are broken down into acetic acid, releasing hydrogen and carbon dioxide. Methanogenesis occurs when methanogenic bacteria produce methane by cleaving two acetic acid molecules to form carbon dioxide and methane (aceticlastic methanogenesis), and/or by reduction of carbon dioxide with hydrogen (hydrogenotrophic methanogenesis).

AD systems adhere to the same basic principles whether the feedstock is food waste, animal manures or wastewater sludge. It will be understood that even though the AD process is the same, the design and management of AD systems will vary based on the feedstock, desired products, and economic/practical constraints. Examples include stand-alone digesters, on-farm digesters, and wastewater treatment plant digesters. The different types and operational modes of digestion systems are described below.

### Mesophilic and Thermophilic Systems

Digesters can be designed to run at different temperature ranges. The temperature ranges are typically 86-1000 F (25-45° C.) for mesophillic systems and 122-140° F. (50-60° C. or above) for thermophilic systems. The advantages of thermophilic systems include faster throughput and biogas production per unit of feedstock and/or volume of digester, and the high temperatures kill higher numbers of pathogenic microorganisms. The disadvantages of thermophilic systems include higher capital costs, higher energy costs for heating, and they generally involve more hands on management. Mesophillic digesters have the advantages of being easier to operate and maintain, but have the disadvantage that they do not result in high pathogen kill.

### Feedstock Variation

Digesters can be designed to process one type of feedstock or to process multiple feedstocks. Digesters can also be designed for co-digestion of more than one type of organic feedstock at the same time. In some embodiment, the feedstock is pre-treated before digestion (e.g., blended, screened, thermally conditioned, etc.).

### Wet (Low Solids) and Dry (High Solids) Digestion Systems

A wet digestion system, also referred to as a low solids AD system, typically processes feedstock with less than 15 percent solids content. Because of the relatively high liquid content, the feedstock for a wet digester is often a slurry that can be mixed or pumped. A dry digester system, also referred to as a high solids AD system, generally processes feedstock with greater than 15 percent solids content. The feedstocks for a dry digester system can be stacked. Dry AD systems are generally less expensive to operate because there is less water to heat and there is more gas production per unit feedstock. However, wet AD systems generally have a lower set-up capital cost.

### Batch Versus Continuous Flow

AD systems can be operated in batch or continuous flow mode. In batch mode, the feedstock is added to the digester all at the same time and there is a set period of time for digestions to occur. Following digestion, the digester is emptied and reloaded with new feedstock. In a continuous flow system, the feedstock is constantly fed into the digester and digested material is continuously removed. Batch digestion systems have the disadvantage of having to open the digester and restart the system every few weeks, which can create operational challenges. Thus, most digesters are continuous flow systems, which also provide more biogas per unit feedstock and lower operating costs. However, dry systems can be operated in batch mode, and highs and lows in gas production can be normalized by using multiple batch digesters with staggered changeover times.

### Single, Double or Multiple Digesters

AD systems can include multiple digesters to ensure each stage occurs sequentially and is as efficient as possible. Multiple digesters can produce more biogas per unit feedstock but at a higher capital cost, higher operating cost, and greater operational management.

### Vertical Tank or Horizontal Plug Flow

Some AD systems comprise one or more vertical tanks that input feedstock through a pipe on one side while digestate overflows through a pipe on the other side. Horizontal plug-flow systems use a solid feedstock (called a ‘plug’) that flows through a horizontal digester at the same rate it is fed into the digester. Vertical tanks have the advantage of being simpler and cheaper to operate, but the disadvantage that feedstock may not reside in the digester for the optimum period of time to produce the most biogas. Horizontal tank systems are more expensive to build and operate, but provide the advantage that the feedstock will reside in the digester for an optimum period of time to produce the most biogas.

### EXAMPLE EMBODIMENTS

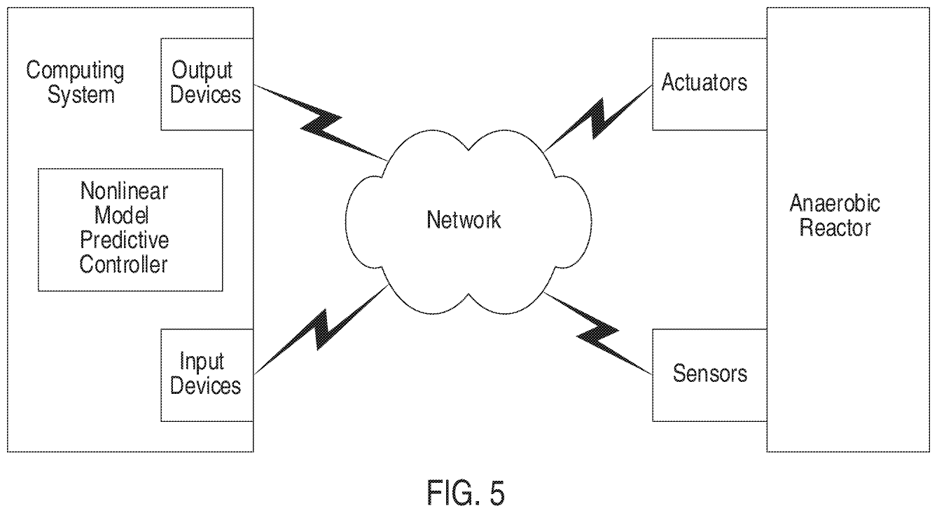

FIG. 5 illustrates an example operating environment in accordance with at least one embodiment. The example operating environment may include an anaerobic digestion reactor. Sensors connected with the anaerobic digestion reactor may send signals to a computing system via a network. The computing system may have input devices configured to receive signals from the sensors. The computing system may maintain a nonlinear model predictive controller configured with a nonlinear model of anaerobic digestion. The computing system may update the nonlinear model predictive controller and/or cause the nonlinear model predictive controller to update. The signals received at the input devices may correspond to input variables of the nonlinear model predictive controller. Updating the nonlinear model predictive controller may update output variables of the nonlinear model predictive controller. Certain output variables of the nonlinear model predictive controller may correspond to actuators of the anaerobic digestion reactor. The computing system may include output devices configured to transmit signals corresponding to output variables of the nonlinear model predictive controller. For example, the output devices may transmit signals to the actuators through the network.

The anaerobic digestion reactor may include any suitable anaerobic digestion equipment. The sensors may include any suitable sensor of anaerobic digestion reactor attributes. Sensors may utilize any suitable signaling protocol to communicate with the computing system. The computing system may utilize any suitable input and/or output devices to receive and/or transmit signals. The computing system may transform and/or decode received signals into nonlinear model predictive controller input variables, and similarly transform and/or encode nonlinear model predictive controller output variables into signals suitable for transmission. The network may include any suitable number of additional control layers (including zero).

The nonlinear model predictive controller may have any suitable number of input and/or output variables. Nonlinear model predictive controller input variables may include input variables from the nonlinear model of anaerobic digestion. Examples of input variables include control variables, manipulated variables, variables corresponding to sensor outputs, variables corresponding to estimated reactor attributes, and variables corresponding to actuator inputs. Examples of output variables include control variables, manipulated variables, and variables corresponding to actuator inputs. The nonlinear model predictive controller may maintain a history of input and/or output variables as well as of derived state variables. The nonlinear model predictive controller may be configured with any suitable nonlinear model (preferably simple in terms of computational efforts and/or of low computational complexity) of anaerobic digestion (e.g. SADM).

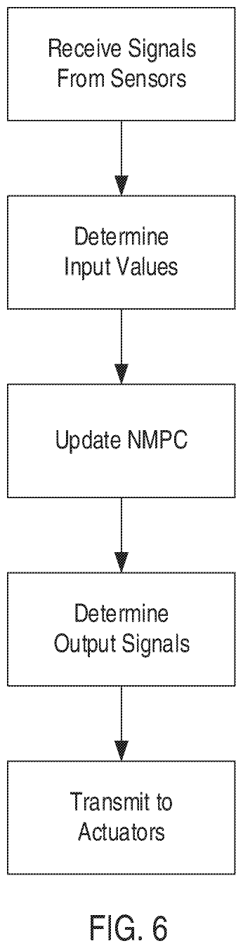

FIG. 6 illustrates an example procedure in accordance with at least one embodiment. Signals may be received from sensors. For example, the input devices may receive the signals generated by sensors. Input values may be determined. For example, values of input variables of the nonlinear model predictive controller may be determined based on the received signals. The nonlinear model predictive controller may update. For example, the nonlinear model predictive controller may update periodically, due to new input data availability and/or as prompted manually. Output signals may be determined. For example, output signals may be determined based on values of output variables of the nonlinear model predictive controller. Signals may be transmitted to actuators. For example, the output signals may be transmitted to the actuator through the network.

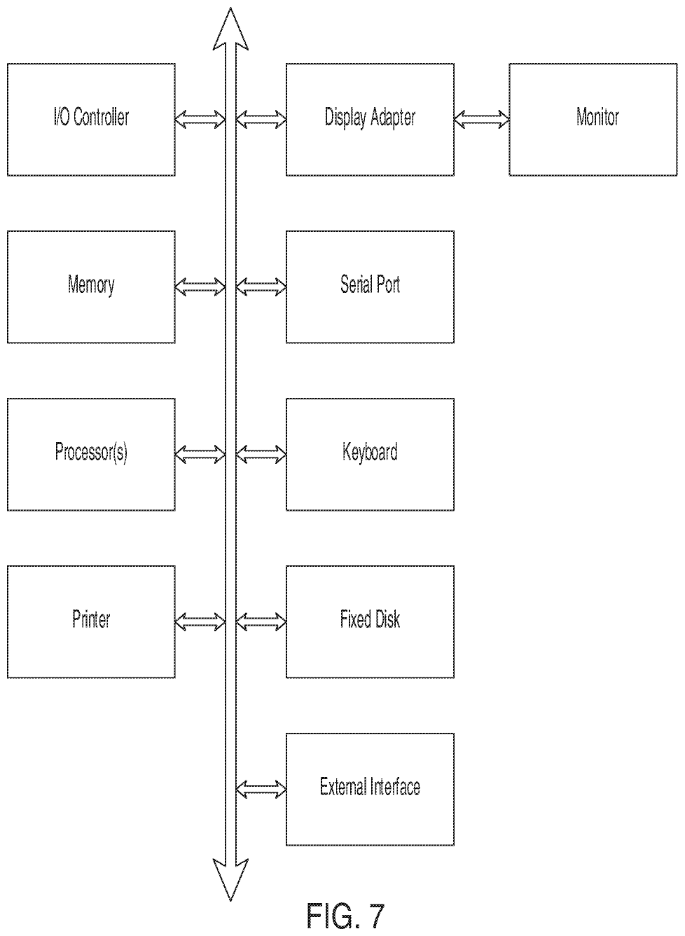

In accordance with at least some embodiments, the system, apparatus, methods, processes and/or operations for message coding may be wholly or partially implemented in the form of a set of instructions executed by one or more programmed computer processors such as a central processing unit (CPU) or microprocessor. Such processors may be incorporated in an apparatus, server, client or other computing device operated by, or in communication with, other components of the system. As an example, FIG. 7 depicts aspects of elements that may be present in a computer device and/or system configured to implement a method and/or process in accordance with some embodiments of the present disclosure. The computer device and/or system of FIG. 7 is an example of a computing system. Further examples of a computing system include a programmable logic controller (PLC). The subsystems shown in FIG. 7 are interconnected via a system bus. Additional subsystems such as a printer, a keyboard, a fixed disk, or a monitor, which is coupled to a display adapter, can be included. Peripherals and input/output (I/O) devices, which couple to an I/O controller, can be connected to the computer system by any number of means known in the art, such as a serial port. For example, the serial port or an external interface can be utilized to connect the computer device to further devices and/or systems not shown in FIG. 7 including a wide area network such as the Internet, a mouse input device, and/or a scanner. The interconnection via the system bus allows one or more processors to communicate with each subsystem and to control the execution of instructions that may be stored in a system memory

and/or the fixed disk, as well as the exchange of information between subsystems. The system memory and/or the fixed disk may embody a tangible computer-readable medium.

It should be understood that one or more of the embodiments described herein can be implemented in the form of control logic using computer software in a modular or integrated manner. Alternatively, or in addition, embodiments may be implemented partially or entirely in hardware, for example, with one or more circuits such as electronic circuits, optical circuits, analog circuits, digital circuits, integrated circuits (“IC”, sometimes called a “chip”) including application-specific ICs (“ASICs”) and field-programmable gate arrays (“FPGAs”), and suitable combinations thereof. As will be apparent to one of skill in the art, notions of computational complexity and computational efficiency may be applied mutatis mutandis to circuits and/or circuitry that implement computations and/or algorithms. Based on the disclosure and teachings provided herein, a person of ordinary skill in the art will know and appreciate other ways and/or methods to implement one or more embodiments described herein using hardware and/or a combination of hardware and software.

Any of the software components, processes or functions described in this application may be implemented as software code to be executed by a processor using any suitable computer language such as, for example, Java, C++ or Perl using, for example, conventional or object-oriented techniques. The software code may be stored as a series of instructions, or commands on a computer readable medium, such as a random access memory (RAM), a read only memory (ROM), a magnetic medium such as a hard-drive or a floppy disk, or an optical medium such as a CD-ROM. Any such computer readable medium may reside on or within a single computational apparatus, and may be present on or within different computational apparatuses within a system or network.

TABLE 1
List of controller tuning parameters, start-up process conditions, CV set-points, and
optimization variables implemented and tested for the proposed NMPC control schemes

| | |
|---|---|
| Sampling Time, Tₛ (h) | 12 |
| Control Horizon, M | 4 sampling time steps (2 days) |
| Prediction Horizon, P | 8 sampling time steps (4 days) |
| Adapted Inoculum Concentration at Start-up (gVSS/L) | 2.78 |
| Organic Substrate (glucose) Feed Characteristics: | |
| Glucose Concentration, S_{S, inf} (M) | 1.45 × 10⁻² |
| | (~2750 mgCOD/L) |
| Organic Substrate Alkalinity (mol HCO₃_{^{−}}/L) | 2.31 × 10⁻³ |
| Organic Substrate pH | 7.50 |
| Nutrient concentrations in organic substrate feed: | |
| Inorganic Nitrogen (M) | 2.07 × 10⁻² |
| Inorganic Carbon (M) | 2.45 × 10⁻³ |
| Cations (M) | 3.32 × 10⁻³ |
| Anions (M) | 2.10 × 10⁻² |
| External Alkali Input Concentration (g NaHCO₃/L) | 16.8 |
| CV Set-points: | |
| S_{ac}^{set} (M) | 1.31 × 10⁻³ |
| | (~84 mgCOD/L) |
| X_{ac}^{set} (M) | 0.188 |
| | (~4.25 gVSS/L) |
| ${r}_{{CH}_{4}}^{set} ( \frac{gCOD}{L . h} )$ | 0.496 |
| Q_{S, LB} (L/h) | 0 |
| Q_{H2O, LB} (L/h) | 0 |
| Q_{alk, LB} (L/h) | 0 |
| vᵤₚ^{min} (m/h) | 0.1 |
| vᵤₚ^{max} (m/h) | 1.5 |
| OLRₘₐₓ (kgCOD/m³.d) | 35 |
| NMPC Objective Function Weights | |
| w₁, w₂ (for both NMPC designs) | 3, 5 respectively |
| w₃ (for NMPC_{Base}) | 5 |
| w₃ (for NMPC_{No rCH4}) | 0 |
| w₄ (for both NMPC designs) | 0.7 gCOD/gNaHCO₃ |

TABLE 2
List of measurements to determine variables and
parameters used in an example NMPC scheme.

| Measurement | Variables Determined |
|---|---|
| Volumetric Flows | Q_{S}, Q_{H2O}, Q_{Alk} (MVs*) |
| Influent & Effluent COD* | S_{S, inf} & S_{S} + S_{ac} (CV1*) |
| Aceticlastic methanogenesis tests, | X_{ac} (CV2*) |
| VSS, COD tests | (48 h delayed measurement |
| | updates used as initial |
| | points for estimator as |
| | described in FIG. 2) |
| Biogas flow, biogas compositions | r_{CH}_{₄} (CV3*) |
| Effluent Partial & Intermediate | [HCO₃⁻], [S_{ac}⁻], Z (total |
| Alkalinities & pH | alkalinity), S_{IC}, & S_{ac} |
| Influent characterisation tests, Total | Stoichiometric/kinetic parameters |
| organic carbon (TOC^{a}), COD^{a} tests, | for organic substrate degradation |
| Influent pH, Influent Partial | |
| Alkalinity | |

*COD—Chemical oxygen demand; MVs—Manipulated variables; CV—control variable; TOC—Total organic carbon
VSS—Volatile suspended solids

TABLE 3
Adopted system conditions from the study of (Subramanyam and
Mishra, 2013) for optimal AD start-up control scenario.

| | Upflow anaerobic sludge |
|---|---|
| AD Reactor System | blanket reactor |
| Reactor Volume (L) | 9.75 |
| Reactor ID (mm) | 100 |
| Temperature (° C.) | 35 |
| Feed | Synthetic (glucose) |
| Volatile suspended solids (VSS) of inoculum | 10.1 |
| (g/L) | |
| Specific methanogenic activity (SMA) of | 0.0625 |
| inoculum on acetate (gCOD-CH4/gVSS · d) | |

TABLE 4
Virtual plant (ADM1) output variables
interface with NMPC and SADM variables.

| | Corresponding |
|---|---|
| | variables in |
| Some of the monitored output variables from | NMPC scheme and |
| virtual plant (ADM1) | SADM |
| S_{ac, eq} (M) = 1/M_{COD, ac}) · (Sᵥₐ · M_{COD, va} + | S_{ac} (M) |
| S_{bu} · M_{COD, bu} + Sₚᵣₒ · M_{COD, pro} + S_{ac} · | (CV and SADM state) |
| M_{COD, ac}) | |
| X_{ac} (M) | X_{ac} (M) |
| | (CV and SADM state) |
| r_{CH}_{₄}_{, total} (gCOD/L · h) = (ρ_{T, ch4} + D · S_{CH}_{₄}) · | r_{CH}_{₄} (gCOD/L · h) |
| M_{COD, CH}_{₄} | (CV and SADM |
| | output) |
| S_{IC} (M) | S_{IC} (M) |
| | (SADM state) |
| Sₛᵤ (M) | S_{S} (t) (M) |
| | (Used in SADM ODE) |
| P_{CO}_{₂} (bar) | P_{CO}_{₂} (bar) |
| | (Used in SADM ODE) |

TABLE 5

| Measurements/Variables | Sensor/Measurement Technique | Mode |
|---|---|---|
| Volumetric flow rates | Flow meters | Online |
| (influent & effluent) | | |
| pH (influent & effluent) | pH meters/probes | Online |
| Temperature | Resistance thermometers, | Online |
| | pyrometers, or thermocouples | |
| Chemical oxygen demand | Lab based analyses (COD kits with | Offline |
| (COD) of influent & | spectrometer for measuring levels | |
| effluent streams | of COD in collected samples) | |
| | UV/vis spectroscopic probes | Online |
| | (research based developments in | |
| | recent years) | |
| Biogas flow rate | Flow meters | Online |
| Biogas composition | Gas analyzers or infrared | Online |
| | absorption measurement based | |
| | sensors | |
| Alkalinities (influent | Titration experiments in lab | Offline |
| & effluent streams) | NIRS*, MIRS*, or FTIRS* based | Online |
| | sensors (research based | |
| | developments in recent years) | |
| Volatile suspended solids | Lab based analyses | Offline |
| (reactor and effluent) | (collection of samples at each | |
| | sampling time) | |
| | NIRS*, MIRS* (research based | Online |
| | developments in recent years) | |
| Aceticlastic methanogenic | Lab based analyses | Offline |
| activity (reactor) | (collection of samples at each | |
| | sampling time) | |
| VFA* concentrations | Titrimetry based instruments or | Online |
| and/or composition | UV/vis spectroscopic probes, | |
| (influent and effluent) | NIRS*, MIRS*, or FTIRS | |
| | (research based developments in | |
| | recent years) | |
| | Gas chromatography, HPLC | Offline |
| | (High performance liquid | |
| | chromatography) | |
| Substrate kinetic tests, | Lab based analyses | Offline |
| total organic/inorganic | | |
| carbon (TOC, TIC) content | | |
| (influent and effluent) | | |

*NIRS—near infrared spectroscopy; MIRS—mid infrared spectroscopy; FTIRS—fourier transform infrared spcectroscopy; VFA—volatile suspended solids.

TABLE 6

| Sensor/Equipment | Variable(s)/Parameter(s) |
|---|---|
| UV/vis spectroscopic probes | COD, TOC, VFA levels |
| Near InfraRed spectroscopy (NIRS) based | VFA levels, Volatile solids, |
| sensors | |
| Mid InfraRed spectroscopy (MIRS) based | VFA levels, total alkalinity, |
| sensors | ammonium, total solids |
| Fourier Transform InfraRed (FTIR) and | VFAs, total and partial |
| fluorescence spectroscopy based sensors | alkalinities, TOC, COD, |
| | sulphates |

TABLE 7
Summary of quantitative results on the performance of the AD system with the tested
start-up operation strategies.

| | | Integral error criteria^{b} | | | Offset Errors^{d} of plant | | |
|---|---|---|---|---|---|---|---|
| | | values of plant | | | outputs relative to set- | | |
| | Time till achieving | outputs (using ITAE^{c} | | | points at steady state | | |
| AD Start-up | steady OLR* of | criterion) | | | (%) | | |
| Operation | 15 kgCOD/m³ · d(d) | S_{ac} | X_{ac} | r_{CH4} | S_{ac} | X_{ac} | r_{CH4} |
| Manual^{a}: 11% pAB | 50 | 5.3 | 282 | 580 | +32 | −3.1 | −3.5 |
| set rate for OLR | | | | | | | |
| Manual^{a}: 25% pAB | 22 | 2.96 | 131 | 353 | +9.6 | −2.4 | −6.6 |
| set rate for OLR | | | | | | | |
| Manual^{a}: 50% pAB | 11 | 4.78 | 180 | 775 | −10 | −3.9 | −19 |
| set rate for OLR | | | | | | | |
| NMPC_{Base} | 39 | 4.43 | 57.2 | 74.5 | +8.8 | +0.37 | +0.23 |
| NMPC_{No rCH4} | 18 | 1.40 | 50.6 | 73.9 | +0.20 | +0.13 | +0.080 |

OLR - Organic loading rate.
^{a}Angelidaki et al. (2006) Method.
^{b}Calculation of the integral error criteria has been conducted for a time duration of 120 days. The integrals were calculated using numerical integration in MATLAB ® R2015b.
^{c}ITAE - Integral of the time-weighted absolute error = ∫₀^{t} t . | setpoint (t) − measurement(t) | . dt.
${}^{d} Offset Error = \frac{Plant {Output}_{steady state} - Set \text{-} point}{Set \text{-} point} \cdot 100.$

### Appendix A. Model Predictive Control (MPC) Basic Principles

The main objective of conventional MPC is to determine a set of manipulated input changes (i.e. MVs) to optimally drive the selected process outputs (i.e. CVs) to their desired set points (Seborg et al., 2011) while considering constraints (if any). These optimisation calculations incorporate process plant measurements and model predicted future outlook of the outputs. FIG. 9 shows the basic mechanism for MPC. At the current sampling time instant (tₖ), using latest available measurements and model for predictions of future outputs, MPC calculates an optimal set of control moves or input values (u*) that can be manipulated (i.e. MVs) within an allowable number of time steps for control, specifically called the control horizon (M). After reaching the control horizon, the input values are fixed at a constant value (equal to the input values determined for the last time step within the control horizon i.e. at t_{k+(M-1)}) for the remaining time steps till the prediction horizon (P). Although a sequence of control moves over the entire prediction horizon (P) is determined, MPC implements only the first time step control moves set. These control calculations are repeated at each sampling time instant. The basic MPC algorithm is summarized as follows (Gme and Pannek, 2011):

At each sampling time instant,
1. Measure outputs and states of the system
2. Using the latest measurements as the initial states, solve the optimal control problem to optimise the desired control objective function over the prediction horizon (P) and consequently, determine the optimal sequence of control inputs (size=number of MVs x control horizon)
3. Select the first time step input set from the sequence and implement it on the process plant being controlled

The adopted process model plays a critical role in the success of MPC since the model is utilized in parallel with MPC calculations. While a linear or linearized model based predictive control (linear MPC) is common, using a nonlinear model is advantageous in case of highly nonlinear systems (Seborg et al., 2011). In this case, the control scheme is said to be a nonlinear MPC (NMPC) scheme.

### Appendix B. Methodology for State Estimation of Aceticlastic Methanogenic Biomass Concentration (X_{ac})

Practically, microbial biomass measurements require long times (days) and hence, the levels of the microbial biomass cannot be monitored online. For control purposes, state estimation is required to estimate the levels of aceticlastic methanogens (X_{ac}); one of the key control variables in the proposed NMPC scheme. For the estimation of X_{ac}, the simple AD model (sADM) had been implemented (same as the model used during NMPC optimizations). The estimation methodology is illustrated in FIG. 8 with an example. At each sampling event, the estimator (sADM) would require the initial state conditions (4 states including X_{ac} for sADM) to solve the model equations. These initial conditions represent the latest available measurements of all the states. The estimator (sADM) is then solved for a time period starting from the time where these measurements are available till the time of the current sampling event. Since X_{ac} measurements require time before obtaining a reading, there is a delay before getting the latest available measurement of X_{ac}. Hence, the start time for the solution of the equation in the estimator (i.e. sADM) depends on the latest available value of X_{ac}. This start time remains same for the upcoming sampling events till the new X_{ac} update becomes available. As seen in FIG. 8, measurement update delay of 2 sampling time periods for X_{ac} is assumed for illustrative purposes. According to the example, the sADM estimations at sampling events 1 and 2 are conducted while considering the initial time (0) as the start time for solutions, where the measurement for X_{ac} was available. However, at sampling event 3, the start time for sADM solution of the estimator is changed to the time of sampling event 1 since the delayed update (after a total duration of 2 sampling time periods) of X_{ac} from sampling event 1 becomes available.

### Appendix C. NMPC Objective Function Terms Values Contribution after Optimisations

FIG. 10 shows the values of the individual cost terms in the overall objective functions of the two proposed NMPC designs (Eq. (11) for NMPC Base and Eq. (19) for NMPC_{No rCH4} in the journal article) after optimisation at each sampling instant for the AD start-up case scenario considered in the journal article. The durations for the control designs in FIG. 11 represent the periods during which the controllers were active.

### Appendix D. Comparison of Observer Estimation and Simple AD Model (sADM) Predictions with Virtual Plant (ADM1) Outputs During NMPC Implementation

FIGS. 11-12 compare the sADM (prediction model during NMPC optimisations) predictions of process variables with those of the virtual plant (modelled via ADM1) during the NMPC scheme implementations (NMPC_{Base} and NMPC_{No rCH4} respectively). FIG. 13 compares the observer estimation of the aceticlastic methanogenic biomass (X_{ac}) CV with the virtual plant output during NMPC implementations. The durations in FIGS. 11-13 represent the time periods during which the NMPC controllers were active.

### Appendix E. NMPC Performance Under Different Scenarios

To evaluate and compare (with each other) the performance of the implemented NMPC designs (NMPC Base and NMPC_{No rCH4}) for robustness and possible improvements, different scenarios were considered during AD start-up control:

### 1. Model Mismatch Bias Correction

At each sampling time instant, the differences between latest plant outputs and the sADM predicted outputs (4 model states and CH₄ production rate) are added as corrections to the sADM predictions during the next NMPC optimization:

Bias_{variable}=Plant measurement_{variable}−sADM prediction_{variable}; at a given sampling instant

Corrected sADM prediction_{variable}=sADM prediction_{variabie}+Bias_{variable}; for next NMPC optimization

### 2. Online Measurement of X_{ac}

In this scenario, it is assumed that direct online measurement of the aceticlastic methanogenic biomass concentration (X_{ac}) is available (i.e. no X_{ac} estimator and hence, all the variables are measured online).

### 3. Instrument Errors

Random errors (as noise) are added to the actual plant output measurements (partial pressure of CO₂, effluent concentration of glucose substrate, effluent pH, variables corresponding to the 4 sADM states, and CH₄ production rate). A random percent change (relative to actual measurement) value between −10% and 10% was generated at each sampling event.

### 4. Measured Disturbances of Glucose Substrate Influent COD Concentration

Disturbance in influent COD of glucose substrate (measured) was initiated at the 10^{th} day and maintained for duration of 15 days. Before and after this disturbance, the influent COD was maintained at the base concentration 2.75 gCOD/L. Two sets of simulations were conducted for each NMPC design: (i) 75% decrease in base influent COD concentration (˜0.69 gCOD/L) as disturbance, and (ii) 75% increase in base influent COD concentration as disturbance (˜4.8 gCOD/L).

### 5. Unmeasured Disturbances of Glucose Substrate Influent COD Concentration

Disturbances in influent COD of glucose substrate are conducted, but these disturbances are unknown to sADM (NMPC prediction model) i.e. the disturbances are unmeasured. Hence, for sADM, the influent COD concentration of the substrate is constant (at the base value of 2.75 gCOD/L) throughout the simulations despite the disturbances. Four sets of simulations were conducted for each NMPC design:
(i) random changes in influent COD ranging between −5% and 5% (relative to base value of influent COD),
(ii) random changes in influent COD ranging between −75% and 75% (relative to base value of influent COD), (iii) 80% decrease in base influent COD concentration (˜ 0.55 gCOD/L), and iv) 80% increase in base influent COD concentration as disturbance (˜ 5.0 gCOD/L). The random disturbances (i)-(ii) occur throughout the duration of operation (simulation time=120 d) whereas the single disturbances (iii)-(iv) are initiated at 10^{th} day for 15 days (for non-disturbance periods, the influent COD is at the base value 2.75 gCOD/L).

Table E and FIGS. 14-31 show the results obtained with the NMPC designs tested under these scenarios (sections E1-E6). For all scenarios, NMPC control and prediction horizons were at 4 and 8 respectively with 12 h sampling.

### E1. Quantitative Summary of Results

Table E summarizes the AD start-up performance with the NMPC designs tested under the different scenarios in terms of quantitative assessments.

TABLE E
Summary of quantitative assessments of the AD start-up performance with the tested NMPC designs
under different scenarios.

| | | | Integral error criteria^{a} values of | | | | | |
|---|---|---|---|---|---|---|---|---|
| | AD Start-up | Time till switching to | plant outputs (using ITAE^{b} | | | Offset Errors^{c} of plant outputs | | |
| | Management | steady OLR* of | criterion) | | | relative to set points at SS* (%) | | |
| Scenario | Scheme | 15 kgCOD/m³.d (d) | S_{ac} | X_{ac} | r_{CH4} | S_{ac} | X_{ac} | r_{CH4} |
| Model Mismatch/Bias | NMPC_{Base} | 4.5 | 1.27 | 111 | 160 | +5.44 | −5.54 | −3.76+ |
| Correction | NMPC_{No rCH4} | 16 | 1.13 | 46.3 | 68.0 | 0.174 | −0.192 | −0.770 |
| Online Measurement of | NMPC_{Base} | Exact OLR not reached | 16.5 | 119 | 210 | +148 | −6.94 | −4.88 |
| X_{ac} | | (OLR at SS* = | | | | | | |
| | | 15.1 kgm³.d) | | | | | | |
| (without X_{ac} estimator) | NMPC_{No rCH4} | 17.5 | 1.50 | 54.4 | 79.6 | +0.177 | −0.201 | −0.774 |
| Instrument Errors | NMPC_{Base} | 15 | 2.16 | 90.2 | 184 | Variable due to fluctuations | | |
| (Random changes ranging | NMPC_{No rCH4} | 13.5 | 1.10 | 95.8 | 188 | Variable due to fluctuations | | |
| between −10% and 10% relative to | | | | | | | | |
| actual measurements) | | | | | | | | |
| Substrate Influent COD | NMPC_{Base} | 54.5 | 6.55 | 134 | 171 | +10.7 | +0.132 | −0.515 |
| Disturbance (Measured) | NMPC_{No rCH4} | 30.5 | 2.54 | 122 | 210 | +0.238 | −0.292 | −0.814 |
| (Decrease of Influent COD to | | | | | | | | |
| 0.69 gCOD/L for 15 days, | | | | | | | | |
| disturbance starting on 10^{th} day) | | | | | | | | |
| Substrate Influent COD | NMPC_{Base} | 10.5 | 150 | 1266 | 2305 | +1588 | −98.6 | −65.8 |
| Disturbance (Measured) | (process | | | | | | | |
| (Increase of Influent COD to | failure) | | | | | | | |
| 4.8 gCOD/L for 15 days, disturbance | NMPC_{No rCH4} | 12.5 | 11.1 | 151 | 120 | +2.74 | +0.174 | −0.459 |
| starting on 10^{th} day) | | | | | | | | |

*OLR - Organic loading rate; SS - Steady state
^{a}Calculation of the integral error criteria has been conducted for a time duration of 120 days The integrals were calculated using numerical integration in MATLAB ® R2015b
^{b}ITAE - Integral of the time-weighted absolute error = ∫₀^{t} t . |setpoint(t) − measurement (t) | . dt
${}^{c} Offset Error = \frac{Plant {Output}_{steady state} - Set \text{-} point}{Set \text{-} point} \cdot 100$ ^{d}Average value from the last set of results (towards end of simulation time) showing similar values when corrected to 3 significant figures.

### REFERENCES

Alvarado-Lassman, A., Sandoval-Ramos, A., Flores-Altamirano, M. G., Vallejo-Cantu, N. A., Mendez-Contreras, J. M., 2010. Strategies for the startup of methanogenic inverse fluidized-bed reactors using colonized particles. Water Environ. Res. 82, 387-391.
Angelidaki, I., Chen, X., Cui, J., Kaparaju, P., Ellegaard, L., 2006. Thermophilic anaerobic digestion of source-sorted organic fraction of household municipal solid waste: Start-up procedure for continuously stirred tank reactor. Water Res. 40, 2621-2628. doi:10.1016/j.watres.2006.05.015
Angenent, L. T., Sung, S., Raskin, L., 2002. Methanogenic population dynamics during startup of a full-scale anaerobic sequencing batch reactor treating swine waste. Water Res. 36, 4648-4654. doi:10.1016/S0043-1354(02)00199-9
Austermann-Haun, U., Seyfried, C. F., Zellner, G., Diekmann, H., 1994. Start-up of anaerobic fixed film reactors: technical aspects. Water Sci. Technol. 29, 297 LP-308.
Batstone, D. J., Keller, J., Angelidaki, I., Kalyuzhnyi, S. V., Pavlostathis, S. G., Rozzi, A., Sanders, W. T., Siegrist, H., Vavilin, V. A., 2002. The IWA Anaerobic Digestion Model No 1 (ADM1). Water Sci. Technol. 45, 65-73. doi:10.2166/wst.2008.678
Bernard, O., Hadj-Sadok, Z., Dochain, D., Genovesi, A., Steyer, J. P., 2001. Dynamical model development and parameter identification for an anaerobic wastewater treatment process. Biotechnol. Bioeng. 75, 424-438. doi:10.1002/bit.10036
Boe, K., Angelidaki, I., 2012. Pilot-scale application of an online VFA sensor for monitoring and control of a manure digester. Water Sci. Technol. 66, 2496-2503. doi:10.2166/wst.2012.498
Borzacconi, L., López, I., Passeggi, M., 2006. Start-up and steady-state results of a full-scale UASB reactor treating malting wastewater. Water Sci. Technol. 54, 261-267. doi:10.2166/wst.2006.515
Cho, Y. T., Young, J. C., Jordan, J. A., Moon, H. M., 2005. Factors affecting measurement of specific methanogenic activity. Water Sci. Technol. 52, 435-440.
de Lemos Chernicharo, C. A., 2007. Anaerobic Reactors. Water Intell. Online 6.
Dong, C., Li, Q., Geng, Z., Wang, H., Wang, Z., 2010. Quick Startup of EGSB Reactor Seeded with Anaerobic Digestion Sludge for the Treatment of Actual Domestic Sewage under Ambient Temperature, in: 2010 4th International Conference on Bioinformatics and Biomedical Engineering. pp. 1-3. doi:10.1109/ICBBE.2010.5515567
Drosg, B., 2013. Process monitoring in biogas plants, IEA Bioenergy. doi:retrieved on 15˜Mar. 2017
Esteves, S., Miltner, M., Fletch, S., 2012. Review and Guide for Monitoring Anaerobic Digestion and Biogas Plants for the Optimisation of Biogas and Biomethane Production.
Falk, H. M., Reichling, P., Andersen, C., Benz, R., 2015. Online monitoring of concentration and dynamics of volatile fatty acids in anaerobic digestion processes with mid-infrared spectroscopy. Bioprocess Biosyst. Eng. 38, 237-249. doi:10.1007/s00449-014-1263-9
Fdéz.-Güelfo, L. A., Álvarez-Gallego, C., Sales Marquez, D., Romero García, L. I., 2010. Start-up of thermophilic-dry anaerobic digestion of OFMSW using adapted modified SEBAC inoculum. Bioresour. Technol. 101, 9031-9039. doi:10.1016/j.biortech.2010.07.021
Fedorovich, V., Lens, P., Kalyuzhnyi, S., 2003. Extension of Anaerobic Digestion Model No. 1 with processes of sulfate reduction. Appl. Biochem. Biotechnol. 109, 33-45. doi:ABAB:109:1-3:33 [pii]
Feitosa Cavalcanti, P. F., Souto Medeiros, E. J., Menezes Silva, J. K., Van Haandel, A., 1999. Excess sludge discharge frequency for UASB reactors. Water Sci. Technol.
Gaida, D., Luis, A., Brito, S., Wolf, C., Bäck, T., Bongards, M., Mcloone, S., 2011. Optimal Control of Biogas Plants using Nonlinear Model Predictive Control. ISSC 2011, Trinity Coll. Dublin.
Gaida, D., Wolf, C., Back, T., Bongards, M., 2012. Nonlinear model predictive substrate feed control of biogas plants, in: 2012 20th Mediterranean Conference on Control and Automation, MED 2012—Conference Proceedings. pp. 652-657. doi:10.1109/MED.2012.6265712
García-Diéguez, C., Molina, F., Fernández, E., Roca, E., 2010. Control of re-startup of anaerobic USBF reactors after short stops. Ind. Eng. Chem. Res. 49, 4748-4755. doi:10.1021/ie901164t
Goberna, M., Gadermaier, M., Franke-Whittle, I. H., García, C., Wett, B., Insam, H., 2015. Start-up strategies in manure-fed biogas reactors: Process parameters and methanogenic communities. Biomass and Bioenergy 75, 46-56. doi:10.1016/j.biombioe.2015.02.003
Goux, X., Calusinska, M., Fossépré, M., Benizri, E., Delfosse, P., 2016. Start-up phase of an anaerobic full-scale farm reactor—Appearance of mesophilic anaerobic conditions and establishment of the methanogenic microbial community. Bioresour. Technol. 212, 217-226. doi:10.1016/j.biortech.2016.04.040
Grüne, L., Pannek, J., 2011. Nonlinear Model Predictive Control: Theory and algorithms, Nonlinear Model Predictive Control. Springer, London. doi:10.1007/978-0-85729-501-9_3
Haugen, F. (Telemark U. C., Bakke, R., Bernt, L., 2014. State Estimation and Model-based Control of a Pilot Anaerobic Digestion Reactor. Control Sci. Eng. 2014, 19.
Hickey, R. F., Wu, W. M., Veiga, M. C., Jones, R., 1991. Start-up, operation, monitoring and control of high-rate anaerobic treatment systems. Water Sci. Technol. 24, 207-255.
Holubar, P., Zani, L., Hager, M., Fr??schl, W., Radak, Z., Braun, R., 2003. Start-up and recovery of a biogas-reactor using a hierarchical neural network-based control tool. J. Chem. Technol. Biotechnol. 78, 847-854. doi:10.1002/jctb.854
Janke, L., Leite, A. F., Nikolausz, M., Radetski, C. M., Nelles, M., Stinner, W., 2016. Comparison of start-up strategies and process performance during semi-continuous anaerobic digestion of sugarcane filter cake co-digested with bagasse. Waste Manag. 48, 199-208. doi:10.1016/j.wasman.2015.11.007
Jimenez, J., Latrille, E., Harmand, J., Robles, A., Ferrer, J., Gaida, D., Wolf, C., Mairet, F., Bernard, O., Alcaraz-Gonzalez, V., Mendez-Acosta, H., Zitomer, D., Totzke, D., Spanjers, H., Jacobi, F., Guwy, A., Dinsdale, R., Premier, G., Mazhegrane, S., Ruiz-Filippi, G., Seco, A., Ribeiro, T., Pauss, A., Steyer, J. P., 2015. Instrumentation and control of anaerobic digestion processes: a review and some research challenges. Rev. Environ. Sci. Biotechnol. doi:10.1007/s11157-015-9382-6
Kil, H., Li, D., Xi, Y., Li, J., 2017. Model predictive control with on-line model identification for anaerobic digestion processes. Biochem. Eng. J. 128, 63-75. doi:10.1016/j.bej.2017.08.004
Lagerkvist, A., Pelkonen, M., Wikstrom, T., 2015. Quick-start of full-scale anaerobic digestion (AD) using aeration. Waste Manag. 38, 102-104. doi:10.1016/j.wasman.2014.12.016
Liang, Y., Qiu, L., Guo, X., Pan, J., Lu, W., Ge, Y., 2017. Start-up performance of chicken manure anaerobic digesters amended with biochar and operated at different temperatures, Nature Environment and Pollution Technology.
Lier, J. B. Van, Mahmoud, N., Zeeman, G., 2008. Anaerobic Wastewater Treatment, Biological Wastewater Treatment: Principles, Modelling and Design. IWA Publishing, London. doi:10.1021/es00154a002
Lloret, E., Pastor, L., Pradas, P., Pascual, J. A., 2013. Semi full-scale thermophilic anaerobic digestion (TAnD) for advanced treatment of sewage sludge: Stabilization process and pathogen reduction. Chem. Eng. J. 232, 42-50. doi:10.1016/j.cej.2013.07.062
Mauky, E., Weinrich, S., Nägele, H.-J., Jacobi, H. F., Liebetrau, J., Nelles, M., 2016. Model Predictive Control for Demand-Driven Biogas Production in Full Scale. Chem. Eng. Technol. 39, 652-664. doi:10.1002/ceat.201500412
Meng, Y., Shen, F., Yuan, H., Zou, D., Liu, Y., Zhu, B., Chufo, A., Jaffar, M., Li, X., 2014. Start-up and operation strategies on the liquefied food waste anaerobic digestion and a full-scale case application. Bioprocess Biosyst. Eng. 37, 2333-2341. doi:10.1007/s00449-014-1211-8
Molina, F., Ruiz-Filippi, G., Garcia, C., Lema, J. M., Roca, E., 2009. Pilot-Scale Validation of a New Sensor for On-Line Analysis of Volatile Fatty Acids and Alkalinity in Anaerobic Wastewater Treatment Plants. Environ. Eng. Sci. 26, 641-649. doi:10.1089/ees.2007.0308
Morel, E., Santamaria, K., Perrier, M., Guiot, S. R., Tartakovsky, B., 2005. Multi-wavelength fluorometry for anaerobic digestion process monitoring. Water Sci. Technol. 52, 465-471.
Nielsen, H. B., Uellendahl, H., Ahring, B. K., 2007. Regulation and optimization of the biogas process: Propionate as a key parameter. Biomass and Bioenergy 31, 820-830. doi:10.1016/j.biombioe.2007.04.004
Ordace, A., Ionescu, C. M., Vannecke, T. P. W., Volcke, I. P. E., Nascu, I., Keyser, R. De, 2012. Predictive control of anaerobic digestion of wastewater sludge. A feasibility study, in: 2012 16th International Conference on System Theory, Control and Computing (ICSTCC). pp. 1-7.
Pandey, P. K., Ndegwa, P. M., Soupir, M. L., Alldredge, J. R., Pitts, M. J., 2011. Efficacies of inocula on the startup of anaerobic reactors treating dairy manure under stirred and unstirred conditions. Biomass and Bioenergy 35, 2705-2720. doi:10.1016/j.biombioe.2011.03.017
Puñal, A., Trevisan, M., Rozzi, A., Lema, J. M., 2000. Influence of C:N ratio on the start-up of up-flow anaerobic filter reactors. Water Res. 34, 2614-2619. doi:10.1016/S0043-1354(00)00161-5
Renard, P., Van Breusegem, V., Nguyen, M.-T, Naveau, H., Nyns, E.-J, 1991. Implementation of an adaptive controller for the startup and steady-state running of a biomethanation process operated in the CSTR mode. Biotechnol. Bioeng. 38, 805-812. doi:10.1002/bit.260380802
Rodríguez, J., Premier, G. C., Dinsdale, R., Guwy, a. J., 2009. An implementation framework for wastewater treatment models requiring a minimum programming expertise. Water Sci. Technol. 59, 367-380. doi:10.2166/wst.2009.870
Rozzi, A., Di Pinto, A. C., Limoni, N., Tomei, M. C., 1994. Start-up and operation of anaerobic digesters with automatic bicarbonate control. Bioresour. Technol. 48, 215-219. doi:10.1016/0960-8524(94)90149-X
Sbarciog, M., Loccufier, M., Vande Wouwer, A., 2012. An optimizing start-up strategy for a bio-methanator. Bioprocess Biosyst. Eng. 35, 565-578. doi:10.1007/s00449-011-0629-5
Sbarciog, M., Vande Wouwer, A., 2014. Start-up of multi-species anaerobic digestion systems: extrapolation of the single species approach. Math. Comput. Model. Dyn. Syst. 20, 87-104. doi:10.1080/13873954.2013.817443
Seborg, D., Edgar, T., Mellicamp, D., Doyle III, F., 2011. Process Dynamics and Control, 3rd ed, John Wiley & Sons. New Jersey. doi:10.1007/s13398-014-0173-7.2
Steyer, P., Bouvier, J. C., Conte, T., Gras, P., Harmand, J., Delgenes, J. P., 2002. On-line measurements of COD, TOC, VFA, total and partial alkalinity in anaerobic digestion processes using infra-red ectrometry. Water Sci. Technol. 45, 133-138.
Subramanyam, R., Mishra, I. M., 2013. Characteristics of methanogenic granules grown on glucose in an upflow anaerobic sludge blanket reactor. Biosyst. Eng. 114, 113-123. doi:10.1016/j.biosystemseng.2012.11.010
Vadlani, P. V, Ramachandran, K. B., 2008. Evaluation of influent medium composition and concentration in a batch reactor for effective startup of UASB reactors. J. Environ. Eng. 134, 1023-1028. doi:10.1061/(ASCE)0733-9372(2008)134:12(1023)
Van Lier, J. B., 2008. High-rate anaerobic wastewater treatment: Diversifying from end-of-the-pipe treatment to resource-oriented conversion techniques. Water Sci. Technol. doi:10.2166/wst.2008.040
Williams, J., Williams, H., Dinsdale, R., Guwy, A., Esteves, S., 2013. Monitoring methanogenic population dynamics in a full-scale anaerobic digester to facilitate operational management. Bioresour. Technol. 140, 234-242. doi:10.1016/j.biortech.2013.04.089
Xue, L., Li, D., Xi, Y., 2015. Nonlinear model predictive control of anaerobic digestion process based on reduced ADM1, in: 2015 10th Asian Control Conference (ASCC). pp. 1-6. doi:10.1109/ASCC.2015.7244539.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A system for controlling a start-up phase of anaerobic digestion reactor operation, the system comprising:
one or more input devices configured at least to receive one or more input signals corresponding to one or more sensors connected with an anaerobic digestion reactor;
one or more output devices configured at least to transmit one or more output signals corresponding to one or more actuators connected with the anaerobic digestion reactor; and
a computing system communicatively connected with the one or more input devices and the one or more output devices, the computing system configured to, at least:
determine, based at least in part on the one or more input signals, one or more values of one or more input variables of a nonlinear model predictive controller, the nonlinear model predictive controller being configured with a nonlinear model of anaerobic digestion having a reduced number of model state variables based at least in part on the one or more input variables that are available due to the one or more input signals;
update the nonlinear model predictive controller based at least in part on the one or more values of the one or more input variables; and
cause the one or more output signals to be generated based at least in part on one or more values of one or more output variables of the nonlinear model predictive controller.

2. A system in accordance with claim 1, wherein the nonlinear model of anaerobic digestion comprises a system of ordinary differential equations, wherein the system of ordinary differential equations is based at least in part on variables corresponding to: an effluent concentration of total acetate from the anaerobic digestion reactor, a concentration of aceticlastic methanogens in the anaerobic digestion reactor, a total alkalinity in the anaerobic digestion reactor, a methane production rate of the anaerobic digestion reactor, an effluent concentration of total inorganic carbon from the anaerobic digestion reactor, an effluent concentration of organic substrate from the anaerobic digestion reactor, and a partial pressure of carbon dioxide in an output of the anaerobic digestion reactor.

3. (canceled)

4. A system in accordance with claim 2, wherein the nonlinear model of anaerobic digestion includes methane production occurring through (i) an aceticlastic methanogenesis pathway, and (ii) a hydrogenotrophic methanogenesis pathway, and the nonlinear model of anaerobic digestion determines total alkalinity from acetate (dissociated), bicarbonate, and hydroxide ions alone.

5. (canceled)

6. A system in accordance with claim 1, wherein the one or more output variables of the nonlinear model predictive controller comprise: a volumetric inflow rate of organic substrate to the anaerobic digestion reactor, a volumetric inflow rate of dilution water to the anaerobic digestion reactor, and a volumetric inflow rate of alkali addition to the anaerobic digestion reactor.

7. A system in accordance with claim 1, wherein an objective function of the nonlinear model predictive controller is based at least in part on: an effluent concentration of volatile fatty acids as acetate from the anaerobic digestion reactor, a concentration of aceticlastic methanogens in the anaerobic digestion reactor, a methane production rate of the anaerobic digestion reactor, and a cost term penalizing an amount of alkali added proportional to a volumetric inflow rate of alkali addition to the anaerobic digestion reactor.

8. A system in accordance with claim 1, wherein the anaerobic digestion reactor comprises a continuous anaerobic digestion reactor with solids retention.

9. A system in accordance with claim 1, wherein the reduced number of model state variables comprises an effluent concentration of total acetate from the anaerobic digestion reactor, a concentration of aceticlastic methanogens in the anaerobic digestion reactor, an effluent concentration of total inorganic carbon from the anaerobic digestion reactor, and a total alkalinity in the anaerobic digestion reactor effluent.

10. One or more computer-readable media collectively having stored thereon computer-executable instructions that, when executed with one or more computing systems, collectively at least:
receive one or more input signals corresponding to one or more sensors connected with an anaerobic digestion reactor;
determine, based at least in part on the one or more input signals, one or more values of one or more input variables of a nonlinear model predictive controller, the nonlinear model predictive controller being configured with a nonlinear model of anaerobic digestion having a reduced number of model state variables based at least in part on the one or more input variables that are available due to the one or more input signals;
update the nonlinear model predictive controller based at least in part on the one or more values of the one or more input variables; and
cause one or more output signals to be generated based at least in part on one or more values of one or more output variables of the nonlinear model predictive controller, the one or more output signals corresponding to one or more actuators connected with the anaerobic digestion reactor.

11. One or more computer-readable media in accordance with claim 10, wherein the nonlinear model of anaerobic digestion comprises a system of ordinary differential equations, wherein the system of ordinary differential equations is based at least in part on variables corresponding to: an effluent concentration of total acetate from the anaerobic digestion reactor, a concentration of aceticlastic methanogens in the anaerobic digestion reactor, a total alkalinity in the anaerobic digestion reactor, a methane production rate of the anaerobic digestion reactor, an effluent concentration of total inorganic carbon from the anaerobic digestion reactor, an effluent concentration of organic substrate from the anaerobic digestion reactor, and a partial pressure of carbon dioxide in an output of the anaerobic digestion reactor.

12. (canceled)

13. One or more computer-readable media in accordance with claim 11, wherein the nonlinear model of anaerobic digestion includes methane production occurring through (i) an aceticlastic methanogenesis pathway and (ii) a hydrogenotrophic methanogenesis pathway, and the nonlinear model of anaerobic digestion determines total alkalinity from acetate (dissociated), bicarbonate, and hydroxide ions alone.

14. (canceled)

15. One or more computer-readable media in accordance with claim 10, wherein the one or more output variables of the nonlinear model predictive controller comprise: a volumetric inflow rate of organic substrate to the anaerobic digestion reactor, a volumetric inflow rate of dilution water to the anaerobic digestion reactor, and a volumetric inflow rate of alkali addition to the anaerobic digestion reactor.

16. One or more computer-readable media in accordance with claim 10, wherein an objective function of the nonlinear model predictive controller is based at least in part on: an effluent concentration of volatile fatty acids as acetate from the anaerobic digestion reactor, a concentration of aceticlastic methanogens in the anaerobic digestion reactor, a methane production rate of the anaerobic digestion reactor, and a cost term penalizing an amount of alkali added proportional to a volumetric inflow rate of alkali addition to the anaerobic digestion reactor.

17. One or more computer-readable media in accordance with claim 10, wherein the anaerobic digestion reactor comprises a continuous anaerobic digestion reactor with solids retention.

18. One or more computer-readable media in accordance with claim 10, wherein the reduced number of model state variables comprises an effluent concentration of total acetate from the anaerobic digestion reactor, a concentration of aceticlastic methanogens in the anaerobic digestion reactor, an effluent concentration of total inorganic carbon from the anaerobic digestion reactor, and a total alkalinity in the anaerobic digestion reactor effluent.

19. A method for controlling a start-up phase of anaerobic digestion reactor operation, the method comprising:
receiving, with one or more input devices, one or more input signals corresponding to one or more sensors connected with an anaerobic digestion reactor
determining, with a computing system, based at least in part on the one or more input signals, one or more values of one or more input variables of a nonlinear model predictive controller, the nonlinear model predictive controller being configured with a nonlinear model of anaerobic digestion having a reduced number of model state variables based at least in part on the one or more input variables that are available due to the one or more input signals;
updating, with the computing system, the nonlinear model predictive controller based at least in part on the one or more values of the one or more input variables; and
causing, with the computing system, one or more output signals to be generated based at least in part on one or more values of one or more output variables of the nonlinear model predictive controller, the one or more output signals corresponding to one or more actuators connected with the anaerobic digestion reactor.

20. A method in accordance with claim 19, wherein the nonlinear model of anaerobic digestion comprises a system of ordinary differential equations, wherein the system of ordinary differential equations is based at least in part on variables corresponding to: an effluent concentration of total acetate from the anaerobic digestion reactor, a concentration of aceticlastic methanogens in the anaerobic digestion reactor, a total alkalinity in the anaerobic digestion reactor, a methane production rate of the anaerobic digestion reactor, an effluent concentration of total inorganic carbon from the anaerobic digestion reactor, an effluent concentration of organic substrate from the anaerobic digestion reactor, and a partial pressure of carbon dioxide in an output of the anaerobic digestion reactor.

21. (canceled)

22. A method in accordance with claim 20, wherein the nonlinear model of anaerobic digestion includes methane production occurring through (i) an aceticlastic methanogenesis pathway and (ii) a hydrogenotrophic methanogenesis pathway, and the nonlinear model of anaerobic digestion determines total alkalinity from acetate (dissociated), bicarbonate, and hydroxide ions alone.

23. (canceled)

24. A method in accordance with claim 19, wherein the one or more output variables of the nonlinear model predictive controller comprise: a volumetric inflow rate of organic substrate to the anaerobic digestion reactor, a volumetric inflow rate of dilution water to the anaerobic digestion reactor, and a volumetric inflow rate of alkali addition to the anaerobic digestion reactor.

25. A method in accordance with claim 19, wherein an objective function of the nonlinear model predictive controller is based at least in part on: an effluent concentration of volatile fatty acids as acetate from the anaerobic digestion reactor, a concentration of aceticlastic methanogens in the anaerobic digestion reactor, a methane production rate of the anaerobic digestion reactor, and a cost term penalizing an amount of alkali added proportional to a volumetric inflow rate of alkali addition to the anaerobic digestion reactor.

26. A method in accordance with claim 19, wherein the anaerobic digestion reactor comprises a continuous anaerobic digestion reactor with solids retention, and wherein the reduced number of model state variables comprises an effluent concentration of total acetate from the anaerobic digestion reactor, a concentration of aceticlastic methanogens in the anaerobic digestion reactor, an effluent concentration of total inorganic carbon from the anaerobic digestion reactor, and a total alkalinity in the anaerobic digestion reactor effluent.

27. (canceled)
